(19) United States
(12) Patent Application Publication
(10) Pub. No.: US 2026/0241352 A1
(43) Pub. Date: Aug. 20, 2026

(54) FLUID MIXING DEVICE

(71) Applicant: Hewlett-Packard Development Company, L.P., Spring, TX (US)

(72) Inventors: Mark A. DeVries, Corvallis, OR (US); John Lahmann, Corvallis, OR (US)

(21) Appl. No.: 19/054,747

(22) Filed: Feb. 14, 2025

(51) Int. Cl. B01F 31/65; B01F 33/501; B01F 35/75; B01F 101/23; B01L 3/00; B01L 7/00

(52) U.S. Cl. CPC B01F 31/651; B01F 33/50112; B01F 35/7543; B01L 3/502761; B01L 7/52; B01F 2101/23; B01F 2215/0431; B01L 2200/04; B01L 2200/0668; B01L 2200/0684; B01L 2200/16; B01L 2300/0654; B01L 2300/087; B01L 2300/18; B01L 2400/0487

(57) ABSTRACT

A diagnostic cartridge includes a fluid mixing device including a first chamber, a second chamber, a protrusion separating the first chamber from the second chamber, and a U-shaped channel extending around a first end of the protrusion and fluidly coupling the first chamber to the second chamber. The diagnostic cartridge further includes a pulse-controlled amplification chamber and a fluidic channel fluidly coupling the pulse-controlled amplification chamber to an opening in the U-shaped channel to supply fluid to the fluid mixing device for mixing and to drain the fluid from the fluid mixing device after mixing.

### BACKGROUND

A presence of nucleic acids (e.g., DNA and RNA) in a biological sample or specimen may be useful in diagnosing patients. For example, a presence or amount of a certain nucleic acid may indicate infection or other illness or disease. Preparation of the biological sample may allow the biological sample to be amplified so that an increased number of the nucleic acids proportional to the starting amount of nucleic acids can be detected. Amplification of the nucleic acids may be performed through a method such as a polymerase chain reaction. Detection of the nucleic acids may indicate a presence, absence, or amount of the nucleic acid present in the biological sample.

### SUMMARY OF THE INVENTION

This disclosure relates to systems and method for detecting and correcting clogs in fluidic channels or lysis chambers of a test cartridge.

At least one aspect relates to a diagnostic cartridge. The diagnostic cartridge includes a fluid mixing device including a first chamber, a second chamber, a protrusion separating the first chamber from the second chamber, and a U-shaped channel extending around a first end of the protrusion and fluidly coupling the first chamber to the second chamber. The diagnostic cartridge further includes a pulse-controlled amplification chamber and a fluidic channel fluidly coupling the pulse-controlled amplification chamber to an opening in the U-shaped channel to supply fluid to the fluid mixing device for mixing and to drain the fluid from the fluid mixing device after mixing.

At least one aspect relates to a method of homogenizing a mixture. The method includes supplying a portion of the mixture to a fluid mixing device through a fluidic channel, the fluid mixing device including a first chamber; a second chamber; a protrusion separating the first chamber from the second chamber; and a U-shaped channel extending around a first end of the protrusion and fluidly coupling the first chamber to the second chamber, the U-shaped channel including an opening fluidly coupled to the fluidic channel. The method further includes pressurizing the first chamber to force the portion of the mixture toward the second chamber through the U-shaped channel, pressurizing the second chamber to force the portion of the mixture toward the first chamber through the U-shaped channel, draining the portion of the mixture from the fluid mixing device to a pulse-controlled amplification chamber through the opening in the U-shaped channel, and passing the portion of the mixture through the pulse-controlled amplification chamber.

At least one aspect relates to a diagnostic cartridge. The diagnostic cartridge includes a pump interface fluidly coupleable to a pump, a fluid mixing device including a first chamber; a second chamber, a protrusion separating the first chamber from the second chamber, and a U-shaped channel extending around a first end of the protrusion and fluidly coupling the first chamber to the second chamber. The diagnostic cartridge further includes a fluidic channel fluidly coupled to a bottom portion of the U-shaped channel to supply fluid to and drain fluid from the fluid mixing device, a pulse-controlled amplification chamber fluidly coupled to the fluidic channel to receive the fluid from the fluid mixing device, a first opening at a top portion of the first chamber; a second opening at a top portion of the second chamber, a first valve positionable to fluidly couple the first opening at the top portion of the first chamber to the pump interface, and a second valve positionable to fluidly couple the second opening at the top portion of the second chamber to the pump interface.

### BRIEF DESCRIPTION OF THE DRAWINGS

These and other aspects and features of the present implementations are depicted by way of example in the figures discussed herein. Present implementations can be directed to, but are not limited to, examples depicted in the figures discussed herein. Thus, this disclosure is not limited to any figure or portion thereof depicted or referenced herein, or any aspect described herein with respect to any figures depicted or referenced herein.

FIG. 1A depicts a fluid mixing device of a diagnostic cartridge, in accordance with example implementations.

FIG. 1B depicts a block diagram of a cartridge and an instrument, in accordance with example implementations.

FIG. 2 depicts a block diagram of a cartridge and an instrument, in accordance with example implementations.

FIG. 3 depicts a method of detecting a presence, absence, or amount of a nucleic acid of interest, in accordance with example implementations.

FIG. 4 depicts a cartridge architecture, in accordance with example implementations.

FIG. 5 depicts a method of detecting a presence, absence, or amount of a nucleic acid of interest, in accordance with example implementations.

FIG. 6 depicts a fluid mixing device of a diagnostic cartridge, in accordance with example implementations.

FIG. 7 depicts a fluid mixing device of a diagnostic cartridge, in accordance with example implementations.

FIG. 8 depicts a perspective view of a portion of the fluid mixing device of FIG. 7.

FIG. 9 depicts the fluid mixing device of FIG. 7 during a mixing process, in accordance with example implementations.

FIG. 10 depicts a method of homogenizing a mixture, in accordance with example implementations.

It will be recognized that the figures are schematic representations of examples for purposes of illustration. The figures are provided for the purpose of illustrating example implementations with the explicit understanding that the figures will not be used to limit the scope of the meaning of the claims. Thus, the description is not limited to the examples and/or implementations provided in the drawings.

### DETAILED DESCRIPTION

In the following detailed description, reference is made to the accompanying drawings which form a part hereof, and in which are shown by way of illustration of specific examples in which the disclosure may be practiced. It is to be understood that other examples may be utilized, and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Biological samples may contain molecules or particles that are of interest. For example, the molecules may be indicative of disease, illness, genetic abnormalities, etc. Molecular diagnostics may be of use to accurately diagnose potential infections or other diseases. Currently, molecular diagnosing may be performed, at least partially, manually. For example, preparing a sample to be analyzed for diagnostic purposes may be performed manually by a human. Manual sample preparation may be slow. Further, a skilled technician may be required to perform the sample preparation. The preparation may be required to be performed in a certified laboratory meeting certain standards or conditions. This may be costly and time-consuming.

Further, molecular detection may be performed by amplifying a molecule of interest (also referred to as a “target” or “target molecule”) in the biological sample and detecting a presence, absence, and/or amount of the molecule of interest. In various examples, the molecule of interest may be a nucleic acid. Specifically, the target molecule may be an oligonucleotide (a “target oligonucleotide”). The target oligonucleotide may be a nucleic acid of interest that is present in and extracted from the biological sample. The target oligonucleotide may be single stranded or double stranded (i.e., before denaturation). The target nucleic acid sequence is the sequence that is amplified. This specific nucleic acid sequence may characterize the presence of a pathogen (e.g., a virus or bacteria) for which the diagnostic method is being used.

One method of amplification may be isothermal amplification (e.g., the entire reaction or reaction chamber is heated and cooled to a uniform temperature). Isothermal reactions may be associated with low-plex reactions (e.g., reactions having a limited number of targets or components), high costs for reagents used in the reactions, and less sensitivity and/or specificity compared to other types of amplification reactions, such as polymerase chain reactions (PCR). Further, sample preparation methods, specifically for magnetized particles used for amplification reactions, may include additional steps. For example, a chaotropic salt/alcohol sample preparation method may include air drying alcohol and eluting a target molecule from the magnetic particles. These additional steps may incur additional costs and may increase an amount of time it takes for diagnosing. An extraction-free sample preparation may be limited by a reaction volume, as high costs of reagents may limit volumes that can be used for a given reaction or analysis. A limited reaction volume may subsequently limit an amount of target analytes that can reach the amplification reaction step(s), thus reducing sensitivity of the results.

The systems and methods described herein provide rapid, low-cost, point-of-care molecular diagnostics. The systems and methods may be convenient to both health care providers running the diagnostics and patients waiting to be diagnosed. The systems and methods described herein utilize pulse-controlled amplification (PCA) to amplify and subsequently detect molecules of interest that may be found in a biological sample. PCA reactions may enable rapid thermocycling with a low power expenditure by using paramagnetic particles to concentrate DNA or other nucleic acid targets into a thin thermocycling zone. Additionally, PCA utilizes polymerase chain reaction (PCR) chemistry to replicate and amplify target molecules (e.g., nucleic acids of interest, such as DNA or RNA). The paramagnetic particles may automate and integrate solid-phase extraction of the sample specimen with PCA and a real-time multi-channel detection, thus reducing both a run time of the diagnostics and a cost of running the diagnostics. Additionally, the diagnostics are performed in a low-cost, disposable cartridge that provides an automated sample-to-answer process. Further, the systems and methods described herein reduce power consumption of instruments used to carry out a multiplex reaction by utilizing low power, yet solid-state and rapid, thermocycling for PCR.

In one example, a cartridge can include a plurality of zones. The cartridge may be inserted into an instrument that helps to facilitate amplification and detection of the nucleic acid of interest. Different zones of the plurality of zones of the cartridge may include different components and/or have different functionalities. Each of the plurality of zones and/or components may be fluidly coupled and/or may fluidly communicate with one another. As used herein, the term “fluidly coupled” means that two or more fluid transmitting volumes are connected directly to one another or are connected to one another by intermediate volumes or spaces such that fluid may flow from one volume into the other volume. This communication may facilitate the steps of the method (e.g., lysing, hybridization, washing, amplification, detection, etc.) to be performed in or on the cartridge to detect and/or analyze nucleic acids of interest, thus reducing the time to reach a diagnosis. For example, a first zone of the plurality of zones may include an input chamber to input a biological sample containing the nucleic acids of interest. A second zone may include one or more extraction chambers, and a third zone may include one or more detection chambers. Additional zones may include, for example, storage for liquid and/or dry reagents (e.g., a wash buffer, a system composition, lyophilized reagents, etc.). A zone may also be used for reconstitution of a master mix used during amplification. The different zones may be coupled via fluid channels. The cartridge can be inserted into an instrument when the process is ready to begin, and the cartridge may only need to be removed from the instrument upon completion of the processes without any external fluid or reagents being added to the process.

The cartridge may further include valves (e.g., pneumatic valves, membrane pinch valves, wax valves, volcano valves, etc.) to permit fluid flow from zone to zone, chamber to chamber, fluid channel to fluid channel, etc. in such a way that fluids and reagents are delivered to the appropriate locations within the cartridge at appropriate times/during appropriate steps of the processes. The instrument may include a valve actuator associated with each of the valves, which may selectively control the valves to open and close, for example, using pneumatic pressure, magnets, solenoids, etc. Further, an instrument interfacing with the cartridge may include a plurality of pumps (e.g., syringe pumps, air cylinder pumps, diaphragm pumps, etc.) to allow fluid flow through the cartridge. Thus, each step that occurs in the process of detecting a molecule of interest, from input of the sample to detection of the amplified molecules, may be performed on the cartridge. Further, all reagents, reactants, and other components used during the processes may be stored in and disposed of on the cartridge. The cartridge used to perform the processes herein may be a single use cartridge. Thus, each component (e.g., reagents) needed for the various reactions and processes may be stored on the cartridge. Wet and/or dry reagents may be stored in blister devices having frangible seals and/or valves that can control flow of the liquids. Further, used or discarded liquids can be transported back to an original storage blister to reduce a need for a waste chamber in the cartridge or removal of waste external to the cartridge.

Furthermore, the systems and methods described herein utilize PCA reactions, which may be PCR-like amplifications. The amplification may be able to be performed rapidly (e.g., within 5 to 10 minutes), thus reducing a total time it takes for a sample to be analyzed and a diagnosis to be made. Further, the systems and methods described herein leverage chemistry associated with PCR reactions for a multiplex reaction (e.g., a reaction that facilitates detecting multiple nucleic acids of interest) and real-time detection. Multiplex reactions allow for the ability to detect and amplify multiple nucleic acids of interest. Further, multiplex reactions may allow for detection and amplification of different types of nucleic acids of interest simultaneously (e.g., while the nucleic acids are in the same chamber or different chambers of the cartridge). This may allow for diagnosis of multiple diseases, infections, etc. with one biological sample, thus reducing time, costs, and labor associated with diagnostics.

As previously stated, real-time detection may reduce analysis times and a time to make a diagnosis based on the analysis results. Further, solid-phase extraction may enhance the sensitivity of the results, through purification and concentration. Solid-phase extraction may also allow more challenging specimen types to be analyzed. Specificity of capture of the target molecules may also be increased through hybridization of the target molecules.

The systems and methods described herein utilize pulse-controlled amplification to amplify molecules (e.g., nucleic acids) of interest that may be found in a biological sample. The biological sample may be lysed (e.g., with a system composition and/or a control substance) to isolate the molecules of interest from the biological sample. In some examples, the molecules of interest are nucleic acids and may be referred to as “nucleic acids of interest.” The nucleic acids of interest may also be referred to herein as “target oligonucleotides,” each of which may have a unique oligonucleotide sequence. The isolated molecules of interest may be hybridized to magnetic particles having attached capture oligonucleotides. The capture oligonucleotides may refer to the oligonucleotides attached to the particles, each having a nucleic acid sequence that is complementary to the “target oligonucleotide.” A capture oligonucleotide may be an oligonucleotide attached to the magnetic particles having a sequence designed to be complementary to the “target oligonucleotide.” The capture oligonucleotide may be a synthetic single-stranded segment of nucleic acid (DNA or RNA). The sequence of the capture oligonucleotide may be designed specifically to match (i.e., complement) the nucleic acid sequence of the target oligonucleotide. In various examples, the capture oligonucleotides may be further used to isolate and amplify the nucleic acid of interest. However, it should be understood that various other methods can be used to capture nucleic acids of interest and subsequently isolate and amplify the nucleic acids of interest in proximity to a heating element.

Hybridization may occur by annealing the molecule of interest with the attached capture oligonucleotides having a complementary sequence. The hybridized molecules of interest and magnetic particles may be amplified to generate an increased or amplified number of molecules of interest via a PCA reaction. The presence, absence, and/or amount of the molecule of interest may be determined using, for example, optical detection.

This disclosure is particularly directed to a system configured to perform a hybridization mixing operation in which a sample collected to be tested for the presence of a particular nucleic acid (e.g., for medical diagnostic purposes) is mixed with a pellet of functionalized paramagnetic particles. The particles may be completely homogenized in the sample in under 90 seconds, and the sample and particles may substantially completely drain from the mixing vessel. In another system, the mixing may be performed by repeatedly moving the fluid between two chambers of a mixing device coupled by a relatively long, narrow channel (e.g., by alternatingly pressurizing one of the channels). However, this design is prone to fluid being trapped in the channel, which may contaminate the sample in subsequent nucleic acid detection processes, reducing the sensitivity and reliability of the detection processes. The design of the mixing device with the long, narrow channel may also cause bubbles to form, which may interfere with the subsequent detection processes. Further, the long, narrow channel may also have a high fluid resistance requiring relatively long mixing cycles, increasing the time required to analyze the sample.

The current disclosure relates to a new design for a hybridization mixing device, which may be a component of a sample test cartridge. In some examples, the mixing device includes two mixing chambers that are separated by a protrusion with a U-shaped channel extending around the end of the protrusion. The U-shaped channel includes a small opening at the bottom to couple the mixing device to a fluidic channel and other components of the cartridge. Each mixing chamber can form a funnel shape that directs fluid toward the opening. The system essentially eliminates the long channel joining the two mixing chambers. The shape and relatively short length of the U-shaped channel can result in improved fluid jetting (e.g., low fluid resistance) when one of the chambers is pressurized and the fluid is forced into the opposite chamber, forming swirling vortices to break up the pellet and accelerate mixing. This can also reduce fluid mixing cycle time can by 60 percent. The short fluid path and common low-point drain can also minimize trapped fluid upon draining of the chambers, reducing trapped fluid volume by 88-100% compared to the other system, and substantially stop bubbles from forming in the fluid. The elimination of bubbles and reduction in residual fluid may improve the reliability and sensitivity of subsequent detection processes.

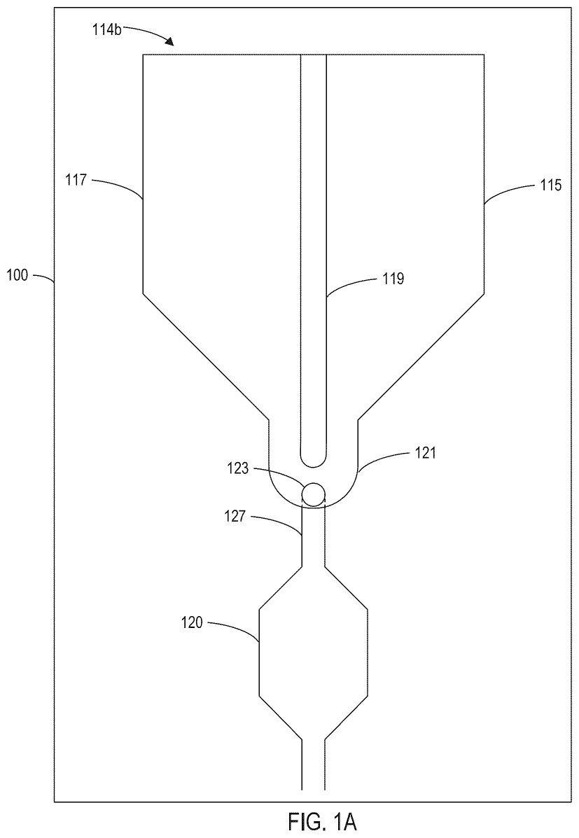

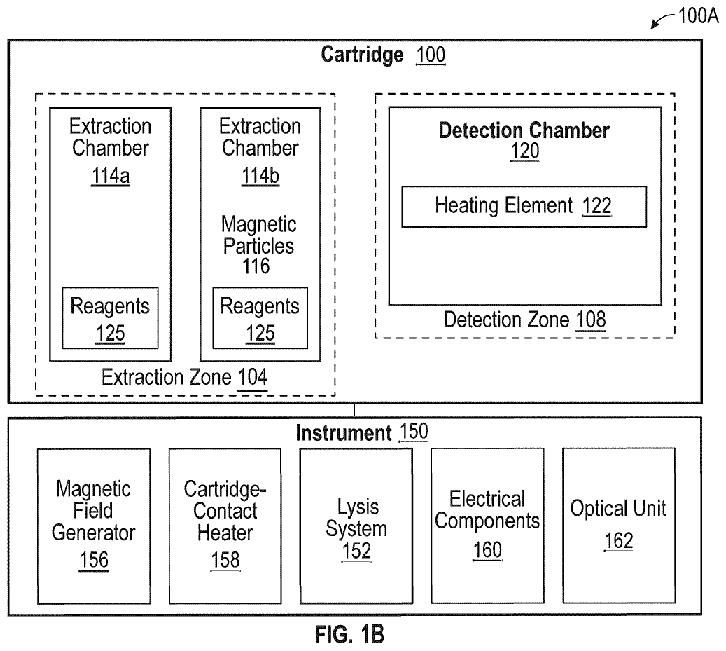

Referring now to FIG. 1A, a portion of a diagnostic cartridge 100 is shown, according to an example embodiment. The cartridge 100 includes a fluid mixing device 114b including a first chamber 115 (e.g., a first hybridization chamber, a first mixing chamber, etc.) and a second chamber 117 (e.g., a second hybridization chamber, a second mixing chamber, etc.) separated by a protrusion 119. The fluid mixing device 114b further includes a U-shaped channel 121 extending around a first end of the protrusion and fluidly coupling the first chamber 115 to the second chamber 117. The U-shaped channel 121 includes an opening 123 fluidly coupling the U-shaped channel 121, and thereby the fluid mixing device 114b, to a fluidic channel 127. The cartridge 100 includes a detection chamber 120 (e.g., a pulse-controlled amplification chamber) fluidly coupled to the opening 123 by the fluidic channel 127.

Fluid may be supplied to the fluid mixing device 114b through the detection chamber 120, the fluidic channel 127 and the opening. As discussed above, one of the chambers 115, 117 may be pressurized to force fluid into the opposite chamber 115, 117 through the U-shaped channel 121. For example, a mixture of sample fluid and buffer fluid may be mixed in a lysis chamber and then supplied to the fluid mixing device 114b. One or both of the chambers 115, 117 may include a pellet of functionalized paramagnetic particles. The functionalized paramagnetic particles may mix with the mixture as the mixture is moved between the chambers 115, 117, for example, until the mixture is homogenized. Both chambers 115, 117 may be pressurized to force the mixture out of the fluid mixing device 114b through the opening 123 and the fluidic channel 127 to the detection chamber 120. The functionalized paramagnetic particles may be captured by a magnet associated with the detection chamber 120, and the rest of the mixture may pass through the detection chamber 120. Additional processes, as described below, may then be conducted using the functionalized paramagnetic particles in order to detect a particular nucleic acid from the sample.

Referring now to FIG. 1B, a diagnostic system 100A, including the cartridge 100 (a diagnostic cartridge) and an instrument 150 is shown, according to an example embodiment. The system 100A may be configured to detect a nucleic acid of interest. In some examples, the cartridge 100 of the diagnostic system may include a series of zones or chambers (e.g., the chambers 115, 117 of the fluid mixing device 114b) that may be defined as cavities. The zones/chambers/cavities may be interconnected via a plurality of channels. The series of cavities/chambers can be individually loaded with reagents. The cavities/chambers can be loaded with the same or with different types of reagents. Loading the cavities/chambers with different types of reagents can permit a multi-step reaction. The reagents/reactants can be disposed in the chambers or blister devices, as will be described herein.

As shown in FIG. 1B, the cartridge 100 includes a first zone 104 comprising a first extraction chamber 114a (e.g., a lysis chamber) having reagents 125 and a fluid mixing device 114b (e.g., a hybridization device), which may include magnetic particles 116 and reagents 125, and a second zone 108 comprising a detection chamber 120 having a heating element 122. The instrument 150 includes a lysis system 152, a magnetic field generator 156, a cartridge-contact heater 158, electrical components 160, and an optical unit 162.

The cartridge 100 may also include a plurality of zones, shown as a first zone 104 and a second zone 108. For example, the first zone 104 may be an extraction zone and the second zone 108 may be a detection zone. Each zone may be or include a plurality of chambers. For example, the first zone 104 may include extraction chamber 114a and/or fluid mixing device 114b and the second zone 108 may include the detection chamber 120. Each zone of the plurality of zones may be in fluid communication with each other (e.g., the other zones of the plurality of zones). It should be understood that the cartridge 100 may include any number of zones. In various examples, each zone is configured to perform or is associated with certain actions. For example, the first zone 104 may be an extraction zone, and the second zone 108 may be a detection zone. Further, each zone may include one or more chambers. For example, the first zone 104 may be an extraction zone including one or more extraction chambers 114, and the second zone 108 may be a detection zone including one or more detection chambers 120.

Fluid within the cartridge 100 may move through a plurality of fluidic channels. The channels may be fluidly coupled via a plurality of fluidic junctions. Transport of fluid through the cartridge 100 may be moderated by a plurality of valves. For example, the cartridge 100 may include a plurality of pinch valves, membrane valves, etc. configured to selectively permit and restrict flow through the fluidic channels. For example, when a valve is closed, fluid movement may be restricted or blocked. The fluidic channels may permit fluid flow from one of the plurality of zones and/or chambers to another zone and/or chamber. For example, fluid may flow from the extraction zone or an extraction chamber to a detection zone or detection chambers. In various examples, the instrument 150 may include a pump to interface with the cartridge 100, specifically the fluid channels of the cartridge 100, to pump air to move air and the fluid through the channels. In various examples, a pressure differential across each valve opening can be applied to transport fluid in and out of each of the plurality of zones, each of the plurality of chambers, each blister device, etc.

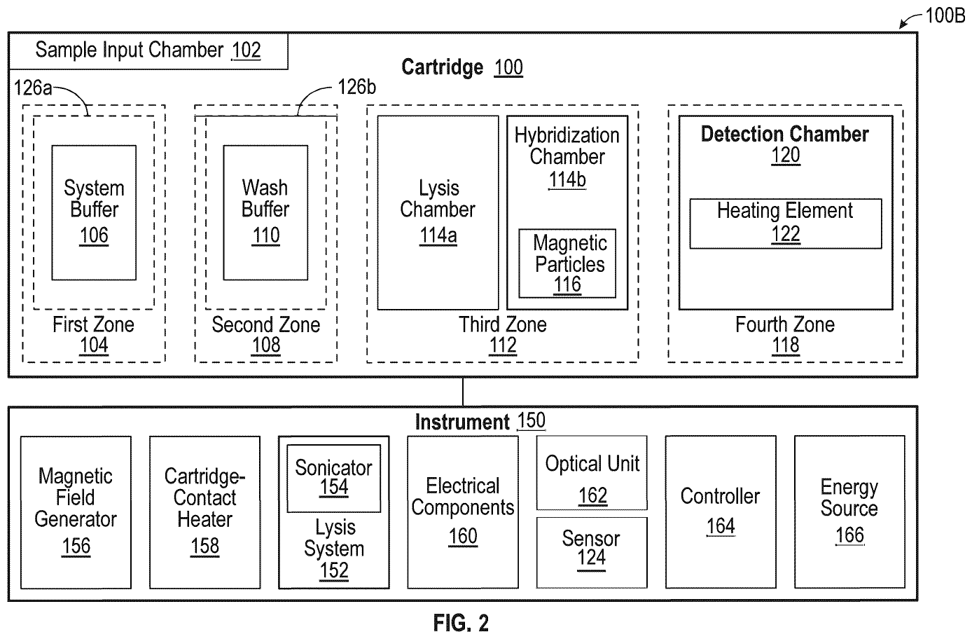

The cartridge 100 may include a greater or fewer number of zones than what is shown in FIG. 1B. For example, as is shown and will be described in FIG. 2, the cartridge 100 may include first through fourth zones 104, 108, 112, and 118. In various examples, different elements of the cartridge 100 may be included in different zones, depending on the configuration of the cartridge 100. For example, as shown in FIG. 2, the cartridge 100 may include a first zone 104 that is an extraction zone having extraction chambers 114 (including the chambers 115, 117 of the fluid mixing device 114b) and a second zone 108 that is a detection zone having the detection chamber 120. In FIG. 2, the first zone 104 includes a system composition 106 and the second zone 108 includes a wash buffer 110. The cartridge 100 of FIG. 2 further includes a third zone 112 having the extraction chambers 114 (e.g., the lysis chamber 114a and the chambers 115, fluid mixing device 114b) and a fourth zone 118 having the detection chamber.

In various examples, at least one of the plurality of extraction chambers 114 may be a lysis chamber 114a. The lysis chamber 114a may house a lysing of the biological sample to release one or more nucleic acids of interest from the biological sample. The lysis chamber 114a may be a fluidic structure with an open cavity or void to define a chamber that could be filled with the biological sample comprising the one or more nucleic acids of interest. The lysis chamber 114a may be configured to house a lysis reaction. For example, a biological sample may enter the lysis chamber 114a and may be lysed, for example by sonication, thermal lysis, thermal sonication, or another lysing method. The lysis reaction may damage the content of the biological sample to release one or more nucleic acids from the biological sample. The lysis reaction performed in the lysis chamber 114a may cause the one or more nucleic acids of interest to be released from the biological sample. The lysis reaction may disrupt, or lyse, cells, and/or tissue samples.

The cartridge 100 may also include one or more reagents 125. The reagents 125 may be located in at least one of the one or more extraction chambers. For example, as shown in FIG. 1B, the extraction chambers 114 include one or more reagents 125, however, it should be understood that the reagents 125 may be stored in any chamber or zone and/or multiple chambers or zones of the cartridge 100. In various examples, the cartridge 100 may include only one reagent in only one chamber (e.g., the detection chamber 120). In various other examples, no reagent 125 may be stored in the extraction chamber 114a, the fluid mixing device 114b or detection chamber 120, and the reagents 125 may be stored elsewhere in the cartridge 100. For example, the reagents may reside in any extraction or detection chambers but may be stored elsewhere instead.

The one or more 125 may be or include liquid reagents 125 and/or dry reagents 125. The reagents 125 may be used in lysing, hybridization, washing, amplification, and/or detection of the nucleic acids of interest. The reagents 125 may also be or include a dry reagent storing an internal positive control (IPC) organism. The reagents 125 may be or include a master mix reagent. The master mix (MM) reagent may be or include a master mix lyophilized (“lyo”) bead of functionalized paramagnetic particles (e.g., magnetic bead (“MB”) lyo particles). The master mix reagent may be used during amplification for reverse transcription and/or amplification and real-time detection of the nucleic acids of interest.

In various examples, liquid reagents 125 may be stored in blister devices (e.g., blister devices 126 of FIG. 2) of the cartridge. For example, liquid reagents (e.g., wash buffer, system composition) may be stored in a metal-lined blister device that may be punctured to release the reagents 125. The one or more reagents 125 may be located in at least one of the one or more extraction chambers 114 or at least one of the one or more detection chambers 120.

In various examples, dry reagents 125 may be stored as lyophilized (e.g., freeze-dried) pellets or cakes, air-dried pellets or cakes, and/or sealed with a plastic plug or film. For example, a dry reagent may be an enzyme used for DNA or RNA elongation during amplification. The dry reagent can be or include master mix or a PCR mixture. The dry reagent 125 may be stored as a pellet. Dry reagents may be dissolved in order to be properly utilized. In various examples, a device may push against a piston to push a liquid out of a blister device to a location of a pellet to dissolve the pellet. The dissolved pellet may then be transported to a desired location (e.g., the detection chamber 120).

In some examples, the lysis chamber 114a includes a heating system. The lysis chamber 114a may be heated to a specific temperature to release the desired molecule of interest from the biological sample (e.g., the nucleic acid of interest). In some other examples, the heating extraction zone or chamber may be a serpentine channel where a fluid of interest having the biological sample is heated during the fluid passage. A serpentine channel may include a series of U-shaped channels that alternate in direction. A serpentine channel may increase a distance the fluid flowing through the channel travels. This may allow ample time to head the fluid to a target temperature.

In various examples, the biological sample can be lysed by a different lysis system, such as a lysis system 152, which may be located within the instrument 150. In various examples, the lysate can be lysed by a plurality of lysing techniques, such as a combination of the lysis system 152, mechanical agitation, an external heat source, ultrasonic agitation, impellors, and/or ceramic or glass beads. In various examples, the lysis system 152 may include a sonicator (shown in FIG. 2 as sonicator 154) to interface with the one or more extraction chambers of the cartridge 100. The sonicator may be used for lysing. The sonicator 154 will be described in greater detail with respect to FIG. 2. The sonicator may also be referred to as a “sonotrode.”

In various examples, at least one of the plurality of extraction chambers 114 may be a fluid mixing device 114b (e.g., the 114b of FIG. 1A). For example, in various embodiments, the extraction zone (e.g., the first zone 104) of the plurality of zones of the cartridge 100 includes at least two extraction chambers 114. As such, two of the extraction chambers 114 may be the chambers 115, 117 (e.g., hybridization chambers) of the fluid mixing device 114b. The fluid mixing device 114b may include a plurality of magnetic particles 116 (e.g., a pellet of functionalized paramagnetic particles in one or both of the chambers 115, 117). The plurality of magnetic particles 116 may include one or more capture oligonucleotides that may be complementary to the one or more nucleic acids of interest. The capture oligonucleotides may be complementary to the nucleic acids of interest because the capture oligonucleotides may be configured and/or selected to bind specifically to (e.g., and only to) the one or more nucleic acids of interest. The fluid mixing device 114b may receive, from the lysis chamber 114a, the one or more nucleic acids released from the lysed biological sample. In the fluid mixing device 114b, the one or more nucleic acids may be hybridized. For example, in the fluid mixing device 114b, the one or more nucleic acids of interest may be hybridized with at least one capture oligonucleotide attached to one or more magnetic particles 116 of the plurality of magnetic particles.

In various examples, the fluid mixing device 114b may include a plurality of hybridization chambers. For example, the fluid mixing device 114b may include two hybridization chambers. The lysate may be moved from the first hybridization chamber to the second hybridization chamber to fully mix and hybridize the nucleic acids of interest.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. For example, hybridization may refer to the formation of a double strand from two single strands, which can each include a nucleic acid and/or a capture oligonucleotide. The capture oligonucleotide may be, for example a DNA or RNA sequence having a complementary sequence to the nucleic acid of interest. Under suitable reaction conditions, the hybridization generally leads to the lowest possible energy state that can be achieved by the combination of the two single strands. In other words, under suitable conditions, the two single strands may bind to each other in such a way that, with respect to the sequences of the two single strands, the greatest possible complementarity (i.e., specificity) is produced.

In some examples, hybridizations are conducted with probe-length nucleic acid molecules, 15-100 nucleotides in length, or 18-50 nucleotides in length. Hybridization and the strength of hybridization (i.e., the strength of the association between the nucleic acids) is influenced by such factors as the degree of complementarity between the nucleic acids, stringency of the conditions involved, and the thermal melting point I of the formed hybrid. The stringency of hybridization conditions may be estimated and/or adjusted such that sequences having at least a desired level of complementarity will stably hybridize, while those having lower complementarity will not. In some examples, specific hybridization occurs under stringent hybridization conditions. An oligonucleotide or polynucleotide (e.g., a probe or a capture oligonucleotide) that is specific for a target nucleic acid will “hybridize” to the target nucleic acid under suitable conditions.

The cartridge 100 may include a plurality of the magnetic particles 116. Specifically, the at least one of the chambers 115, 117 of the fluid mixing device 114b may include a plurality of the magnetic particles 116. The magnetic particles 116 may also be referred to as “magnetic beads,” “beads,” and/or “particles” As used herein, “magnetic beads” or “magnetic particles” refer to microparticles that have ferromagnetic or paramagnetic properties. For example, the magnetic particles 116 may be or include (strept)avidin with biotinylated oligonucleotides, covalently bound oligonucleotides, polymer beads with embedded iron particles, etc. The size of the microparticles is optionally in a range from approximately 10 nm to approximately 2 mm, optionally in a range from 100 nm to 1 mm, optionally in a range from 500 nm to 50 μm. The shape of the microparticles can be freely selected and can, for example, be spherical, cube-shaped, cuboid, or ellipsoidal. The magnetic particles with ferromagnetic properties are optionally formed from at least one of the following materials or contain at least one of the following materials: iron, nickel, cobalt, AlNiCo, SmCo, Nd2Fei4B, NieoFe2o (“Permalloy”), and/or NiFeCo alloys. Optionally, the magnetic particles with paramagnetic properties are made of at least one of the following materials formed or contain at least one of the following materials: alkaline earth metals, alkali metals, and/or rare earths. Alternatively, a magnetic microparticle can be formed from a non-magnetic material such as glass and/or silicate, with magnetic substances being embedded therein. For example, such a microparticle can have a core made of magnetic materials. The magnetic microparticles are optionally provided with at least one coating in order to enable or promote functionalization with nucleic acids, in particular with extraction nucleic acids and/or capture oligonucleotides. Optionally, at least one extraction nucleic acid and/or one capture oligonucleotides and a maximum of 1012 extraction nucleic acids and/or capture oligonucleotides are functionalized on a microparticle. Optionally, an areal density of extraction nucleic acids and/or capture oligonucleotides that are functionalized on the surface of a magnetic microparticle is in a range from 0.0001 to 1 per square nanometer. The microparticles can optionally have a coating which enables and/or facilitates functionalization with capture oligonucleotides. For example, the surface of the magnetic microparticles can be at least partially functionalized with streptavidin.

The magnetic particles 116 may have a nucleic acid attached. Specifically, the attached nucleic acids may be attached to the magnetic particles 116 by capture oligonucleotides that are attached to the magnetic particles 116. The capture oligonucleotides attached to the magnetic particles 116 are selected to be complementary in part or in full to the one or more nucleic acids of interest. In the fluid mixing device 114b, the one or more nucleic acids from the lysis chamber 114a may be hybridized to the capture oligonucleotides attached to the magnetic particles 116. In some examples, the magnetic particles 116 comprise one or more capture oligonucleotides that bind to the one or more nucleic acids of interest (e.g., from the lysis chamber 114a). In various examples, the capture oligonucleotides may be the same or different from each other. For example, a first capture oligonucleotides attached to a first magnetic particle 116 may have a first sequence, and a second capture oligonucleotides attached to a second magnetic particle 116 may have a second sequence.

In various examples, the fluid mixing device 114b includes a plurality of hybridization chambers (e.g., chambers 115, 117 as shown in FIG. 1A). During hybridization, the fluid containing the biological sample may move between the plurality of hybridization chambers to allow for mixing (e.g., partial mixing, complete mixing, etc.) of the solution or fluid during hybridization. Mixing the fluid may occur to distribute the magnetic particles 116 throughout the fluid (e.g., the lysate). Mixing may additionally or alternatively occur using one or more of ultrasonic mixing, acoustic mixing, impellors, mechanical agitation, diffusion, and/or channel geometry, such as turns, ridges, and/or mini chambers.

During hybridization, the fluid may also be heated. The solution may be heated in a variety of ways, such as using an external contact heat source, ultrasonic energy, acoustic energy, and/or infrared radiation (IR).

The second zone 108 may be, in various examples, a detection zone including one or more detection chambers 120 (e.g., a pulse controlled amplification chamber). In various examples, a different zone may include the one or more detection chambers (e.g., the fourth zone 118, as shown in FIG. 2). The detection chambers 120 may not be a part of or associated with a zone.

The cartridge 100 may include one or more detection chambers 120. The detection chambers 120 may be configured to amplify and detect the one or more nucleic acids of interest that have been lysed (e.g., from the biological sample) and hybridized (e.g., to the functionalized magnetic particles 116). Thus, in various embodiments, at least one of the one or more detection chambers 120 is an amplification chamber. The detection chambers 120 may amplify the nucleic acid of interest through, for example, pulse controlled amplification (PCA). PCA is described in greater detail with respect to FIG. 1B0.

As used herein, the terms “amplify” or “amplification” with respect to nucleic acid sequences, refer to methods that increase the representation of a population of nucleic acid sequences in a sample. Copies of a particular target nucleic acid sequence generated in vitro in an amplification reaction may be referred to as “amplicons” or “amplification products.” In various examples, amplification products may refer to any products amplified during an amplification reaction. For example, amplification products may include nucleic acids, unquenched fluorophores generated during each replication cycle of the amplification reaction, etc. Amplification may be exponential or linear. A target nucleic acid may be DNA (such as, for example, genomic DNA and complementary DNA (cDNA) or RNA). While the methods described hereinafter relate to amplification using polymerase chain reaction (PCR), numerous other methods such as isothermal methods, rolling circle methods, etc., may be used either in place of, or together with, PCR methods. In at least one of the detection chambers 120, a lyophilized master mix reagent may be stored. The master mix reagent may refer to a reagent used during an amplification reaction, such as a PCR or PCA reaction.

In various examples, the amplification reactions may utilize primers to perform amplification of the nucleic acids. Primers are short, artificial, single-stranded segments of nucleic acid (e.g., DNA) that are designed to be complementary to the beginning and/or end of the target sequence that will be amplified. The primer sequences may be shorter than the one of the capture oligonucleotides. For example, a primer may contain about 10 to 25 nucleotides.

Primers may perform specific functions during amplification. For example, during the amplification/elongation step of the PCR, the primers may bind to both ends of the nucleic acid of interest (e.g., the DNA sequence of interest), thus bookending the sequence of interest that need to be amplified. Enzymes (e.g., DNA polymerase) may then copy the part of the target oligonucleotide sequence that falls between the primers, selectively amplifying the sequence of interest. In some examples, the capture oligonucleotides described above may also be used as primers during amplification. In other examples, the primers may be oligonucleotides different than the capture oligonucleotides. In some examples, the primers may be forward and/or reverse primers. Forward and reverse primers may denote a direction of elongation during the polymerization by the polymerase enzyme. The primers may be used during the amplification/elongation step of the reaction and may comprise part of the master mix composition described herein.

The detection chamber 120 may include a heating element 122. For example, a detection zone of a plurality of zones may include one or more detection chambers 120 having one or more heating elements 122. Each detection chamber 120 may include a heating element 122. In various examples, at least one of the one or more detection chambers may be an amplification chamber. Further, the heating element of the amplification chamber may be a foil configured to interact with the electrical components 160 of the instrument 150. The electrical components 160 may provide an electrical connection between the instrument 150 and the detection chamber 120, thus providing heat modulation to the detection chamber 120 to amplify the one or more nucleic acids of interest that have been hybridized to the capture oligonucleotides of the plurality of magnetic particles. Specifically, the electrical connection may activate the one or more heating elements 122 of each of the one or more detection chambers 120.

The heating element 122 may be positioned or located at a side portion (e.g., only a single side, such as a single wall of the detection chamber 120) of the detection chamber 120. The heating element 122 may include a plurality of layers comprising at least a foil. The heating element 122 may be attached to one or more of an adhesive and/or a heat spreader or conductor. The heating element 122 may be or include, in various examples, a continuous, structured, or shaped metal foil, metal wires, a conductor and/or resistor layer deposited and/or plated, and/or backed by a heat spreader. The heating element 122 may be configured to heat the detection chamber 120 during the PCA process. The heating element 122 may be, for example, a foil to locally heat a portion of the detection chamber 120. For example, a heating element 122 of the amplification chamber (e.g., the detection chamber 120) may be or include a foil to interact with electrical components 160 of the instrument 150 provide an electrical connection to activate the one or more heating elements 122 of each of the one or more detection chambers 120. The heating element 122 may provide heat modulation to amplify the one or more nucleic acids of interest that have been hybridized to the at least one capture oligonucleotide attached to one or more magnetic particles 116 of the plurality of magnetic particles in the fluid mixing device 114b.

During the PCA reaction occurring in the detection chamber 120, the heating element 122 may be used to heat a portion of the volume of the detection chamber 120. For example, only a portion of the detection chamber 120 proximate the heating element 122 may be heated. The heating element 122 may be heated by a short electric pulse or other process. Each pulse may be short (e.g., between about 10 and about 1200 microseconds) and each pulse may be delivered quickly in succession. In some examples, the heating time in at least one amplification cycle, in at least 10, at least 20, at least 40, at least 80, or in at least 160 amplification cycles is more than 1 nanosecond, more than 5 nanoseconds, or more than 10 nanoseconds and less than 100 milliseconds, less than 10 milliseconds, less than 1 millisecond, less than 300 microseconds, less than 100 microseconds, less than 50 microseconds, less than 30 microseconds, less than 10 microseconds, less than 5 microseconds, or less than 1.5 microseconds.

Due to a resistance of the foil and the rapid pulsing, heat created by the pulses may be local (e.g., the pulses heat only a small portion of the detection chamber 120 surrounding the heating element). In various examples, the pulses may be generated by an energy or power source (e.g., energy source 166). When the pulses are ceased or removed, the portion of the detection chamber 120 that has been heated by the pulses may return to the same temperature as the rest of the detection chamber not proximate the heating element 122. As such, denaturation of the nucleic acids or other molecules occurs in a small volume near the heating element 122.

Denaturation of a nucleic acid may include separate it into its two single strands. For example, the original can be separated from the complement during denaturing. Denaturing may also be referred to as melting. The denaturing of the nucleic acid double strand may be thermally induced. For example, at least a part of the nucleic acid double strand or the whole double strand is exposed to a temperature, described as a denaturing temperature, which causes or at least encourages a separation of the nucleic acid double strands. The denaturing temperature may not be a fixed temperature but may be a temperature interval, within which the temperature during denaturing varies. The denaturing temperature may be selected to be so high that nucleic acid double strands can be separated and/or so low that a DNA polymerase, which may aid in denaturation, is not substantially damaged. In some examples, a denaturation temperature may be between 90 and 100 degrees Celsius. For example, a denaturing temperature may be 95 degrees Celsius.

This heating process may cause the overall temperature of the detection chamber 120 to be isothermal (e.g., no change, substantially no change, or minimal change in temperature is seen during heating for the PCA reaction), because the heated portion of the detection chamber 120 is small enough that an overall temperature change is not seen by the detection chamber 120. In various implementations, heat flow in and out of the detection chamber 120 may be present. The heating element 122 may cause a slight (e.g., less than 1 degree Celsius) increase in the overall temperature of the detection chamber 120. For example, heat may be unable to be transferred out of the detection chamber 120, and each pulse generated by the heating element 122 may add heat to the chamber. However, the pulse may heat a small enough volume of the detection chamber 120 relative to the total volume of the detection chamber 120 such that the volume heated by the heating element 122 can rapidly return to a setpoint temperature of the detection chamber 120.

Referring still to FIG. 1B, the instrument 150 is shown, according to example embodiments. The diagnostic system 100A may include the instrument 150 to interface with (e.g., connect to) the cartridge 100. The cartridge 100 may be inserted into the instrument 150 to perform the processes described herein. In various examples, the cartridge 100 may be a single use cartridge. For example, one cartridge may be used one time to detect the presence of nucleic acids in one biological sample.

The instrument 150 may include a lysis system 152. The lysis system 152 may interface with at least one of the one or more extraction chambers 114 of the cartridge 100 (e.g., the lysis chamber 114a). The lysis system 152 may include a sonicator 154 (shown in FIG. 2). The sonicator 154 may interface with the at least one extraction chamber 114 (e.g., the lysis chamber 114a). The sonicator 154 may include an ultrasonic horn that transmits ultrasonic waves generated by the sonicator 154 into a sample in the lysis chamber 114a to agitate the fluid in the lysis chamber 114a. The lysis system 152 may be utilized when the biological sample enters the extraction chamber 114a of the cartridge 100 to lyse the sample and release the nucleic acids of interest. The lysis system 152 may be coupled to or positioned proximate the lysis chamber 114a when the cartridge 100 is inserted into the instrument 150. For example, the ultrasonic horn may be positioned immediately adjacent the extraction chamber 114a such that ultrasonic waves transmitted by the ultrasonic horn may propagate through a sample in the lysis chamber 114a.

In various examples, the lysis system 152 may be a lysis system comprising sonication (e.g., use of ultrasonic energy), thermal lysis, and/or thermal sonication system. In some examples, the lysis system 152 may include a heating system. In examples where the lysis system 152 includes a heating system, the lysis system 152 may heat the extraction chamber 114a to a target temperature (e.g., between 35 and 100 degrees Celsius), such as by heating the heating element of the lysis chamber 114a to the target temperature, to release the desired molecule of interest (e.g., nucleic acid) from the biological sample. For example, the lysis system 152 may heat the lysis chamber 114a to 95 degrees Celsius. In some embodiments, the sonicator 154 may generate heat in a sample in the lysis chamber 114a using ultrasonic waves.

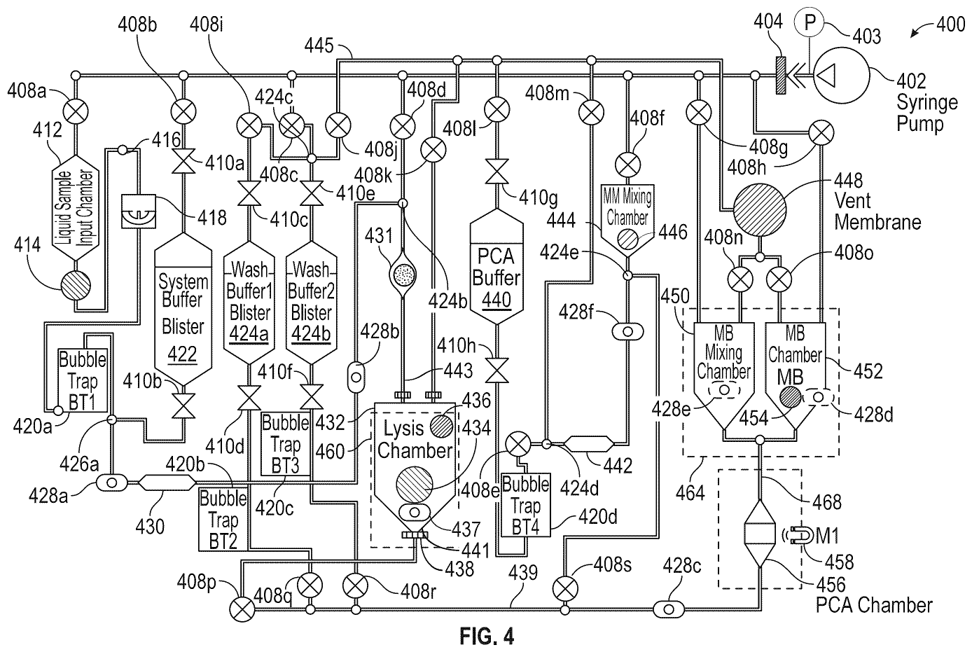

In some examples, the lysis system 152 is programmable. Thus, a user may be able to control, set, determine, etc. lysing protocol parameters (e.g., using the controller 164), such as the sample volume, sonication power level, acoustic frequency, and lysing duration. The lysis system 152 may also provide a cooling feature, enabled by a heat exchanging sub-assembly, which may prevent the biological sample from exceeding a maximum set temperature during operation. The lysis system 152 is discussed below in further detail with respect to FIG. 4.

The instrument 150 may include a magnetic field generator 156 to generate a magnetic field. The magnetic field may be used to dock the plurality of magnetic particles 116 to at least one zone of the plurality of zones of the cartridge 100. In various examples, the magnetic field generator 156 is a magnet (e.g., a permanent magnet). The magnetic field generator 156 may be positioned at or proximate the detection chamber 120. The magnetic field generator may be configured to generate a magnetic field such that the magnetic particles 116 that are hybridized with the nucleic acid of interest and/or the capture oligonucleotides are separated from the lysate solution such that the PCA reaction can occur. The magnetic field generator 156 may be a current carrying conductor or other device that may be powered on or activated such that electric charges being moving to create the magnetic field. In various embodiments, the instrument 150 may be activated or powered on to activate the magnetic field generator.

In some examples, the magnetic field generator 156 is configured to generate a variable magnetic field in such a manner that acts on at least a part of the magnetic particles 116 present in the detection chamber and that are linked to the nucleic acid of interest.

In another example, the variable magnetic field may act on at least a part of the magnetic particles 116 present in the detection chamber 120 in such a manner that the magnetic particles 116 attach to the local heating element 122. Further, the variable magnetic field may be configured to act on the magnetic particles 116 attached to the local heating element 122 in such a manner that they leave the local heating element 122 and are suspended in a reaction solution. This may allow the magnetic microparticles 116 to be optionally attached to and/or repelled from the local heating element 122 multiple times, allowing them to hybridize with additional target nucleic acids in the reaction solution.

The magnet or plurality of magnets may be or include a permanent magnet and/or an electromagnet that can be changed in position and/or orientation relative to the reaction container. For example, when using a permanent magnet, the magnetic field may be changed by changing an orientation of the permanent magnet to the detection chamber 120 and/or a distance of the permanent magnet from the detection chamber 120. The direction of the magnetic field can also be changed, for example, by reversing the permanent magnet relative to the reaction container such that, for example, the side of the permanent magnet facing the reaction container changes from the magnetic north pole to the south pole of the permanent magnet or vice versa. When using an electromagnet, for example, the variable magnetic field can be changed by changing the current flow, in such a manner as the current intensity and/or the direction of the current flow. For example, the electromagnet may comprise one or more solenoid coils and optionally a ferromagnetic core. The magnet may be formed on a side of the local heating element 122 facing away from the detection chamber 120. This may offer the advantage that the permanent magnet in this arrangement makes it particularly effective and easy to attract the magnetic microparticles 116 to the local heating element. Alternatively or additionally, the one or more magnets may be changed in position relative to the detection chamber to provide a variable magnetic field in the reaction solution. Alternatively or additionally, several magnets with different polarity can be brought to the detection chamber to achieve a variable magnetic field in the detection chamber.

The instrument 150 may include one or more cartridge-contact heaters 158. The cartridge-contact heater 158 may be a heater configured to heat one or more components, zones, etc. of the cartridge 100. For example, the cartridge-contact heater 158 may heat the extraction chamber 114a during lysing of the biological sample with the system composition and the internal positive control (IPC). The cartridge-contact heater 158 may also be used to heat the lysate during hybridization. In various examples, the one or more heating elements 122 may be or include the cartridge-contact heater 158. The cartridge-contact heater 158 may interface with a heat spreader 5. The heat spreader may be a conduit of the cartridge contact heater 158 and may facilitate heat transfer from the cartridge-contact heater 158 to one or more of the detection chambers 120.

The instrument 150 may include electrical components 160. The electrical components 160 may provide an electrical connection between the instrument 150 and one or more zones, chambers, components, etc. of the cartridge 100. For example, the electrical components 160 may provide an electrical connection between the instrument 150 and the cartridge 100 to activate the one or more heating elements 122 of each of the one or more detection chambers 120. An interaction of the detection chamber 120 and the electrical components 160, via an electrical connection, may provide heat modulation to amplify the one or more nucleic acids of interest that have been hybridized to the capture oligonucleotides of the plurality of magnetic particles 116. The electrical components may provide a connection to the heating element 122 in the detection chamber.

The instrument 150 may include an optical unit 162. The optical unit 162 may be coupled with at least one of the one or more detection chambers. The optical unit 162 may be configured to detect a presence, absence, amount, etc. of the nucleic acid of interest after amplification has been performed. In various examples, the optical unit 162 is to detect a plurality of amplification products indicative of a presence, absence, or amount of the amplified one or more nucleic acids of interest. The optical unit 162 may detect the nucleic acid via a label bound to the nucleic acid of interest during amplification.

In various examples, the cartridge 100 may include a transparent window in at least one detection chamber (e.g., the detection chamber 120) of the one or more detection chambers to allow the optical unit 162 to detect a plurality of amplification products indicative of a presence, absence, or amount of the plurality of amplified nucleic acids of interest. In various examples, the detection chamber 120 may include the transparent window. A transparent window may permit light from the optical unit 162 to pass through the cartridge 100 and/or detection chamber 120 so that light can enter the detection chamber 120 and detect the amplification products indicative of a presence, absence, or amount of the plurality of amplified nucleic acids of interest.

In some examples, detection by the optical unit 162 may be performed in the detection chamber 120 and may be done using a label bound to the nucleic of interest. Specifically, the optical unit 162 may be used for fluorescence sensing. For example, fluorophores may be generated during each replication cycle of the amplification reaction. Fluorophores may be used to infer the presence or amount of nucleic acids in the detection chamber 120. In PCA (and/or PCR), fluorophores may be attached to capture oligonucleotides and a quencher molecule prior to amplification. This compound molecule may be included in the master mix reagent. In the proximity of the quencher, the compound molecule may not fluoresce. However, during PCA (and/or PCR), the compound molecule may anneal or attach to a portion of the target nucleic acids. A polymerase enzyme, while constructing a double stranded amplicon, may release the fluorophore to separate the fluorophore from the quencher and allow the fluorophore to fluoresce.

In some examples, the detection step may include detecting the fluorophore via a sensor located within the optical unit 162. For example, the optical unit 162 and any sensors located within may be positioned in the instrument 150 such that the optical unit 162 and/or the sensors interface with and/or are located proximate the detection chamber 120 when the cartridge 100 is inserted into the instrument 150. In some examples, the sensor may be a fluorescence sensor coupled to the optical unit 162 and configured to detect the fluorescence from one or more of the plurality of zones of the cartridge 100 (e.g., the fourth zone 118, within the detection chamber 120, etc.).

In some examples, the optical unit 162 may detect the nucleic acid of interest by detecting an intensity of fluorescent probes in the detection chamber 120. The detection chamber 120 may be configured to transmit fluorescent emission to a sensor within the optical unit 162 located in the instrument. For example, during amplification, fluorophores may be released (e.g., using Taqman probes) into solution during elongation for specific detection of amplified products. The optical unit 162 may detect the fluorophores that may be indicative of the presence, absence, amount, etc. of the nucleic acid of interest.

Referring now to FIG. 2, a diagnostic system 100B is shown, according to an example embodiment. The system 100B includes a cartridge 100 and an instrument 150. The cartridge 100 and the instrument 150 may include the same or similar elements as the elements found in the cartridge and instrument of the system 100A. For example, the extraction chamber 114a, the fluid mixing device 114b, and the detection chamber 120 may be the same as those described with respect to FIG. 1B.

In addition to the elements described above with respect to FIG. 1B, the cartridge 100 of the diagnostic system 100B includes a sample input chamber 102, a first zone 104 comprising a system composition 106, a second zone 108 comprising a wash buffer 110, a plurality of blister devices 126a-b (individually described as blister device 126, and together described as blister devices 126), a third zone 112 comprising the extraction chamber 114a (e.g., a lysis chamber) and the fluid mixing device 114b (including the hybridization chambers) housing magnetic particles 116 (also referred to herein as magnetic beads and/or beads), and a fourth zone 118 comprising the detection chamber 120 having the heating element 122. The instrument 150 includes the lysis system 152 having a sonicator 154, the magnetic field generator 156, the cartridge-contact heater 158, electrical components 160, the optical unit 162 having a sensor 124, a controller 164 (e.g., a computer including a processor and memory configured to control various aspects of the diagnostic system 100B), and an energy source 166.

The cartridge 100 can include a sample input chamber 102. The sample input chamber 102 may be a chamber or cavity in which a biological sample can be inserted so that the biological sample enters the cartridge 100 and can be analyzed. For example, the cartridge 100 may be configured to receive a biological sample. The sample input chamber 102 may include a liquid port for receiving liquids. The biological sample may be, for example, saliva, blood, cells, etc. In various examples, the biological sample may include one or more nucleic acids. Of the one or more nucleic acids, the biological sample may include one or more nucleic acids of interest. The nucleic acids of interest may be nucleic acids that the cartridge 100 is being used to amplify and analyze. In various examples, the nucleic acids of interest may not be included in a biological sample. For example, the nucleic acids of interest may be isolated and input into the sample input chamber 102. The nucleic acids of interest may be, for example, DNA, RNA, mRNA, etc. The one or more nucleic acids of interest may be the same targets or different targets (e.g., the cartridge 100 can be used to detect and/or analyze one or more of the same type of nucleic acid of interest or different nucleic acids of interest). For example, the diagnostic system 100A of 100B may be configured to amplify multiple nucleic acids of interest to, for example, detect multiple infections, diseases, etc. Amplification and identification of these multiple nucleic acids may be performed simultaneously, thus reducing an amount of time and cost of detecting the presence of multiple nucleic acids.

The cartridge 100 may also include a plurality of zones, shown as a first zone 104, a second zone 108, a third zone 112, and a fourth zone 118. In various examples, one or more of the first zone 104, the second zone 108, and/or the third zone 112 may be an extraction zone. In various examples, the fourth zone 118 may be a detection zone. Each zone may be or include a plurality of chambers. For example, one or more of the first zone 104, the second zone 108, and/or the third zone 112 may include one or more extraction chambers and the fourth zone 118 may include one or more detection chambers. Each zone of the plurality of zones may be in fluid communication with each other (e.g., the other zones of the plurality of zones). It should be understood that the cartridge 100 may include any number of zones. In various examples, each zone is configured to perform or is associated with certain actions. For example, the first zone 104 may be configured to receive and/or house a system composition 106. Further, the first zone 104 may be configured to deliver the system composition 106 to various other components and/or zones of the cartridge 100 to rinse or wash the materials from the respective zones. In various examples, the plurality of zones may include an extraction zone and a detection zone. Further, each zone may include one or more chambers. For example, the plurality of zones may include an extraction zone having one or more extraction chambers and a detection zone having one or more detection chambers.

The system composition 106 may be a buffer or composition useful for lysis and hybridization of a sample (e.g., of the biological sample). The system composition 106 may also be referred to as a “system buffer.” The system composition 106 may include ingredients (e.g., a buffer) to control a pH of the solution. In some examples, the system composition 106 can comprise water, a salt and/or a surfactant. In some examples, the salt is MgCl2, NaCl, KCl, or (NH4)2SO4. In some examples, the system composition 106 comprises between 50 mM and 1M salt. In some examples, the system composition 106 comprises between about 0.001% and about 0.1% (e.g., about 0.001%, 0.01%, 0.05%, or 0.1%) surfactant. In some examples, the surfactant is selected from Tween® 20 (polysorbate 20), Tween® 80 (polysorbate 80), Tween® 85 (polysorbate 85), SPAN® 80 (sorbitan monooleate) or SPAN® 85 (sorbitane trioleate). In some examples, the surfactant is Tween® 20. The system composition 106 may also include a buffering component to buffer the pH of the solution. For example, the buffering component may be a tris buffer.

Each zone may include one or more chambers configured to perform specific actions. As shown in FIG. 2, the first zone 104 may include the system composition 106, the second zone 108 may include a wash buffer 110, the third zone 112 may include a plurality of extraction chambers 114, shown as lysis chamber 114a and fluid mixing device 114b, and the fourth zone 118 may include one or more detection chambers 120. In various examples, each zone may include different types of chambers, different numbers of chambers, different configurations of chambers, etc. In addition to or alternative to chambers, each zone of the plurality of zones may include elements such as blister devices storing solutions (e.g., reagents, buffer solutions, etc.), bubble traps, seals, conduits through which fluid flows from one zone to one or more other zones.

The first zone 104 may include the system composition 106. It should be understood that the system composition 106 may be included in a zone different than the first zone 104. In various examples, the system composition 106 is not included in a zone. The system 100A may include one or more system compositions 106.

As shown in FIG. 2, the second zone 108 may include the wash buffer 110. It should be understood that the wash buffer 110 may be included in a zone different than the second zone 108. In various examples, the wash buffer 110 is not included in a zone. The system 100A may include one or more wash buffers 110. In various examples, the cartridge 100 may include a chamber in a first zone of a plurality of zones to store the wash buffer 110. The wash buffer 110 may be transported from the chamber to at least one detection chamber 120 to remove undesired elements of the biological sample when the biological sample is in the detection chamber 120. In various embodiments, the extraction chamber 114a, the fluid mixing device 114b, and/or the detection chamber 120 may be washed to remove undesirable elements from the chamber being washed.

The wash buffer 110 refers to a buffer used for washing magnetic particles 116 and the detection chamber 120. In some examples, the wash buffer 110 can comprise water, a salt, a buffering compound, or component (e.g., tris buffer) and/or a surfactant. In some examples, the salt is KCl, MgCl2 NaCl, etc. In some examples, the wash buffer 110 comprises between 20 mM and 45 mM salt. In some examples, the wash buffer can comprise between about 0.001% and about 0.1% (e.g., about 0.001%, 0.01%, 0.05%, or 0.1%) surfactant. In some examples, the surfactant is selected from Tween® 20, Tween® 80, Tween® 85, SPAN® 80 or SPAN® 85. In some examples, the surfactant is Tween® 20.

The system 100B may include a plurality of wash buffers 110. Each wash buffer 110 may have a different composition (e.g., one buffer can be or include salt while another buffer can be or include water). For example, a first wash buffer 110 may have a more aggressive wash chemical relative to a second wash buffer. A more aggressive wash chemical may be a harsher chemical that can remove a greater number of undesired elements in the sample of fluid relative to a less aggressive wash chemical, such as because the more aggressive wash chemical can have a higher chemical concentration, pH level, a higher specificity for removing the undesired elements, etc. Thus, the first wash buffer may be used to wash the detection chamber 120 and the second wash buffer may be used to rinse the detection chamber 120. For example, the first wash buffer may be introduced to a chamber (e.g., the detection chamber 120, the extraction chamber 114a, etc.) to wash or remove undesired elements of the sample. Undesired elements may be, for example: remaining sample fluid that is not the nucleic acid of interest, contaminants, or other elements or molecules that may interfere with use of the extracted nucleic acid of interest. The second wash buffer may be introduced to the chamber after the first wash buffer has exited the chamber and the wash has been completed. The rinse may remove any remaining elements or contaminants not removed by the first wash buffer.

In various examples, one or more components or liquid reagents 125 (e.g., the system composition 106 and/or the wash buffer 110) may be stored, housed, or otherwise contained in a blister device 126. The blister devices 126 may store liquid reagents (e.g., the system composition 106, the wash buffer 110, liquid reagents used for amplification, etc.). The blister device 126 may allow fluid to be released so that the fluid can reach a destination (e.g., the detection chamber 120). In various examples, the cartridge 100 may be configured such that the system composition 106 and/or the wash buffer 110 may return to the blister device 126 upon completion of use. For example, the wash buffer 110 may be released from a blister device 126 storing the wash buffer 110 and may be delivered to the detection chamber 120 to wash the contents of the detection chamber. After washing is complete, the wash buffer 110 may return to the blister device 126 to be stored.

The blister device 126 can be configured to transport its contents in a direction according to a selection of a pumping direction through the blister device 126. In some examples, the blister device 126 is configured to transport contents of the blister device 126 in a direction from a first valve toward a second valve. In some other examples, the blister device 126 is configured to transport contents of the blister device 126 in a direction from the second valve toward the first valve. Blister devices may also be referred to herein as blisters. The blister device(s) 126, when present in the cartridge 100, may be or be part of the first zone and/or the second zone of the plurality of zones.

The blister device 126 may comprise a chamber (e.g., a storage cavity) to store a reagent, a first actuator at a first end of the zone, and/or a second actuator at a second end of the zone opposite to the first end of the zone. The blister device 126 can be configured to be hermetically sealed-off from the channel(s) of the cartridge 100 when the diagnostic device/system is in a non-activated state.

The third zone 112 may be, in various examples, an extraction zone including one or more extraction chambers 114, shown as lysis chamber 114a and the hybridization chambers of the fluid mixing device 114b. In various examples, a different zone may include the one or more extraction chambers (e.g., the first zone 104). The extraction chambers 114 may be configured to interact with and/or prepare the biological sample for amplification and detection in the detection chamber 120. In various examples, the extraction chambers 114 may be, for example, lysis chambers 114a, the hybridization chambers of the fluid mixing device 114b, and/or any other types of extraction chambers.

In various examples, the biological sample may be lysed with the system composition 106 and an internal positive control (IPC). The resulting solution of the biological sample, the system composition 106, and the IPC may be referred to as a lysate. The IPC may be included in the lysate to control false negative results. During amplification, the IPC may be amplified with the nucleic acids of interest to indicate that the solution being amplified is functional and a negative result (e.g., the nucleic acids of interest are not detected) is reliable. The IPC may be a lyophilized organism or synthetic organism. In various examples, the IPC may be a protein or organism present in a human sample. Further, the lysis chamber 114a may include a plurality of beads (e.g., glass beads, ceramic beads) used to mechanically agitate or lyse the organisms present in the lysate.

During hybridization, the fluid may also be heated. The solution may be heated in a variety of ways, such as using an external contact heat source, ultrasonic energy, acoustic energy, and/or infrared radiation (IR).

The fourth zone 118 may be, in various examples, a detection zone including one or more detection chambers 120. In various examples, a different zone may include the one or more detection chambers (e.g., the second zone 108). The detection chambers 120 may not be a part of or associated with a zone.

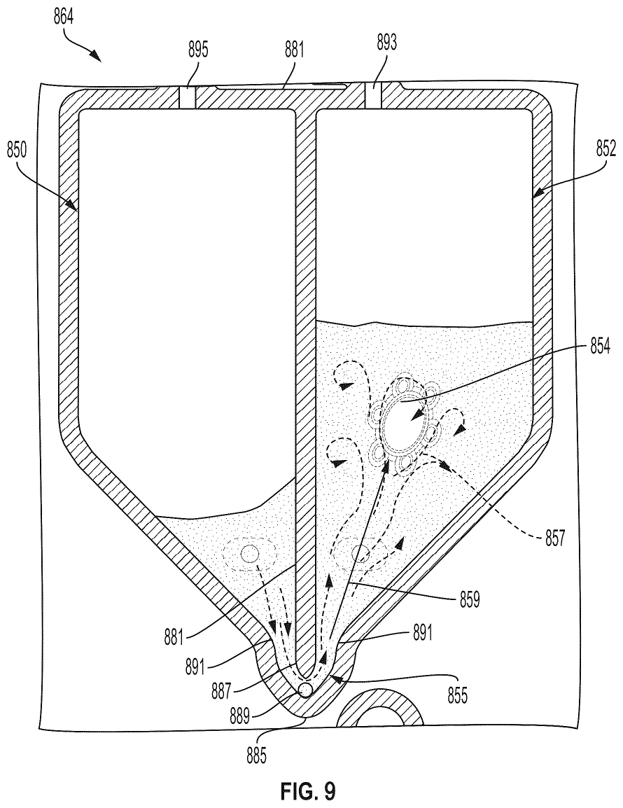

The cartridge 100 may include one or more detection chambers 120. The detection chambers 120 may be configured to amplify and detect the one or more nucleic acids of interest that have been lysed (e.g., from the biological sample) and hybridized (e.g., to the functionalized magnetic particles 116). Thus, in various embodiments, at least one of the one or more detection chambers 120 is an amplification chamber. The detection chambers 120 may amplify the nucleic acid of interest through, for example, pulse controlled amplification (PCA). PCA is described in greater detail with respect to FIG. 9.

Referring still to FIG. 2, the instrument 150 is shown, according to example embodiments. The diagnostic system 100A may include the instrument 150 to interface with (e.g., connect to) the cartridge 100. The components of the instrument 150 may be positioned such that specific components are located proximate to specific corresponding components of the cartridge 100 when the cartridge 100 is inserted into the instrument 150. The cartridge 100 may be inserted into the instrument 150 to perform the processes described herein. In various examples, the cartridge 100 may be a single use cartridge. For example, one cartridge 100 may be used one time to detect the presence of nucleic acids in one biological sample.

The instrument 150 of FIG. 2 may include the lysis system 152, the magnetic field generator 156, the cartridge-contact heater 158, the electrical components 160, and the optical unit 162 described above with respect to FIG. 1B. The instrument 150 of FIG. 2 may further include a controller 164, an energy source 166, and sensors 124.

As stated above, the lysis system 152 may include a sonicator 154. The sonicator 154 may interface with the at least one extraction chamber 114 (e.g., the extraction chamber 114a). The sonicator 154 may deliver ultrasonic waves to the extraction chamber 114a to lyse the fluid present in the chamber. In some examples, the biological sample may be lysed using a sonication system, by using a sonotrode (e.g., the sonicator 154). The lysis system 152 of the instrument 150 may therefore include an ultrasonic transducer or sonicator 154 that transmits ultrasonic energy to the extraction chamber (e.g., the extraction chamber 114a) into the biological sample to cause cell/spore/tissue disruption. Efficient transfer of the ultrasonic energy from the sonicator 154 to the sample within the extraction chamber 114 may be dependent, at least in part, upon maintaining the contact between the transducer tip (e.g., an ultrasonic horn) of the sonicator 154 present in the instrument and the cartridge according to a predetermined force.

The instrument 150 may include electrical components 160 that provide a connection to the heating element 122 in the detection chamber. Additionally, the instrument 150 may include a controller 164 and/or an energy or energy source 166. The energy or power source may generate pulses of energy to locally heat the detection chamber 120 via heat the heating element 122 during PCA. The controller 164 may control or actuate the generation of the electrical pulses delivered by the energy source 166. In various examples, the controller 164 and/or the energy source 166 may be located within the cartridge 100 (e.g., within and/or coupled with the detection chamber 120).

The instrument 150 may also include a plurality of sensors 124 detect fluid movement throughout the cartridge 100. The sensors 124 may detect fluid movement via, for example, reflection. The sensors 124 may be capacitive sensors configured to detect a dielectric property in the plurality of detection chambers 120 and/or one or more of the plurality of zones. In various examples, the sensors 124 may be or include optical sensors configured to detect a dielectric property in one of more of the plurality of detection chambers 120 and/or one or more of the plurality of zones.

The plurality of sensors 124 may be located at various positions throughout the instrument 150. For example, one or more sensors 124 may be positioned within the instrument such that one or more sensors 124 interface with each zone of the plurality of zones of the cartridge 100, each chamber of the cartridge 100, an inlet and/or an outlet of each zone of the plurality of zones, etc. In various examples, the sensors may communicate information to another component of the instrument 150 (e.g., the controller 164) to control one or more components of the cartridge 100. For example, a sensor 124 may receive an indication that fluid has exited a component of the cartridge 100. The sensor 124 may communicate the information to the controller 164, and the controller 164 may responsively close a valve of the cartridge 100 to prevent fluid flow.

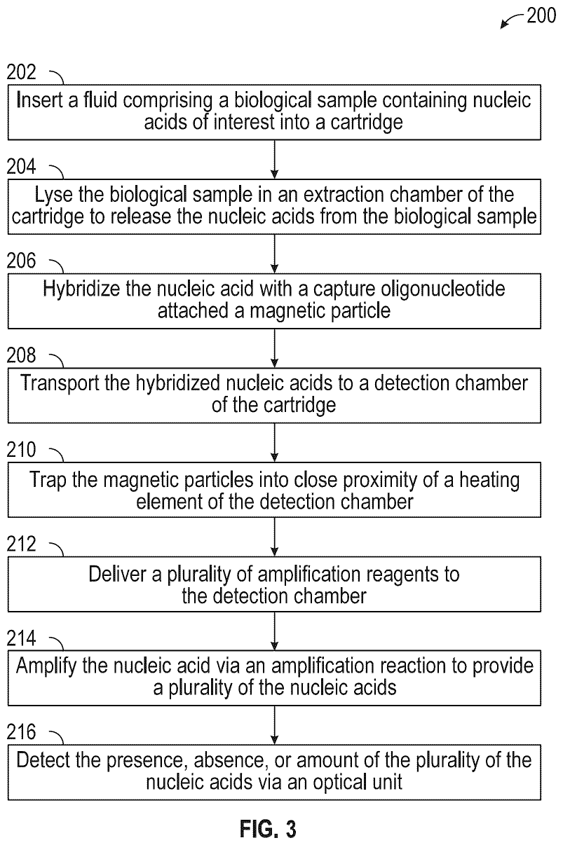

Referring now to FIG. 3, a method 200 for detecting a presence, absence, or amount of a nucleic acid of interest is shown, according to an example embodiment. At process 202, a fluid comprising a biological sample is inserted into a cartridge (e.g., the cartridge 100). In various examples, the biological sample may include the nucleic acid of interest. In various examples, the biological sample may contain another non-nucleic acid molecule of interest. In various examples, the nucleic acid of interest may be a DNA strand and/or an RNA strand. The cartridge may include a plurality of zones. Each zone of the plurality of zones may be in fluid communication with each other. For example, the plurality of zones may be or include an extraction zone and/or a detection zone in fluid communication with one another. The extraction zone may include one or more extraction chambers 114 and the detection zone may include one or more detection chambers 120. In various examples, each detection chamber 120 may include one or more heating elements 122. The cartridge may further include one or more reagents 125 and a plurality of magnetic particles 116.

In various examples, prior to lysing the biological sample at process 204, a system composition (e.g., the system composition 106) may be added to the biological sample. The system composition may comprise water, a salt, and a surfactant. The system composition may be used during the lysing step occurring at process 204.

At process 204, the biological sample is lysed into at least one of the one or more extraction chambers (e.g., extraction chambers 114a). Lysing the biological sample may release the one or more nucleic acids of interest from the biological sample. In various examples, the biological sample may be lysed by, for example, sonication (e.g., using the sonicator 154), heating, mechanical agitation, etc. In various examples, the biological sample may be mixed with an IPC and/or a system composition 106 to form a lysate, which may then be lysed to release the nucleic acids of interest from the biological sample. For example, the method can include lysing in the device a biological sample can include the one or more nucleic acids of interest to release the one or more nucleic acids of interest from the biological sample. For example, the biological sample is lysed by one or more of sonication, thermal lysis, or thermal sonication.

At process 206, the one or more nucleic acids of interest are hybridized in the fluid mixing device 114b. For example, the fluid mixing device 114b may include two fluidly coupled chambers 115, 117 that may be alternatingly pressurized to move fluid between the chambers 115, 117. The one or more nucleic acids of interest may be hybridized to or with at least one capture oligonucleotide attached to one or more magnetic particles 116 of the plurality of magnetic particles 116. For example, the fluid mixing device 114b may include a plurality of magnetic particles 116 within one or both of the chambers 115, 117. The magnetic particles 116 may include attached capture oligonucleotides. The nucleic acids of interest may be hybridized to the capture oligonucleotides.

At process 208, the one or more nucleic acids of interest with the at least one capture oligonucleotide attached to the one or more magnetic particles 116 may be transported to at least one detection chamber 120 of the one or more detection chambers 120. The nucleic acids of interest may be transported, for example, via one or more channels within the cartridge 100.

At process 210, the one or more magnetic particles 116 may be trapped into close proximity of the one or more heating elements 122 of the at least one detection chamber 120. In various examples, the detection chamber 120 may be locally heated at or around the heating element 122 to perform amplification. As such, the magnetic particles containing the nucleic acids of interest may be trapped near the heating element 122 so that the nucleic acids of interest can undergo the amplification reaction to be identified by the optical unit 162.

At process 212, a plurality of amplification reagents 125 may be delivered to the at least one detection chamber 120. In various examples, the plurality of amplification reagents 125 may include, for example, a lyophilized master mix reagent, an enzyme for use in amplification, etc. The amplification reagents 125 may be located in various locations of the cartridge (e.g., in blister devices 126) and may be transported to the detection chamber prior to amplification. In various examples, the amplification reagents may be stored as dry or wet reagents. Dry reagents may be dissolved prior to use.

The method 200 may further include washing or removing, using at least one wash buffer 110, undesired elements from the at least one detection chamber 120. Washing may be performed by introducing the wash buffer 110 into the at least one detection chamber 120. For example, the wash buffer 110 may be released from a blister device 126 and may be transported to the detection chamber 120. A plurality of wash buffers may be used to wash the detection chamber 120.

At process 214, the one or more nucleic acids of interest may be amplified in at least one of the plurality of zones. The one or more nucleic acids of interest may be amplified via an amplification reaction. The amplification reaction may provide a plurality of the one or more nucleic acids of interest.

In various examples, amplifying the one or more nucleic acids of interest may be performed in the at least one detection chamber 120 by generating a pulse of current via an electrical connection of an instrument 150. The instrument 150 may receive and interact with the cartridge 100. The pulse of current may modulate a temperature proximate the one or more heating elements 122 of the at least one detection chamber 120 to increase a temperature proximate the one or more heating elements of the at least one detection chamber 120 to between 90 and 110 degrees Celsius. For example, the temperature may be increased to 100 degrees Celsius. In various examples, only a portion of the detection chamber may be heated by the pulses. In various examples, amplification may be performed by a PCA reaction.

At process 216, a plurality of amplification products indicative of the presence, absence, or amount of the plurality of amplified nucleic acids of interest may be detected. The amplification products may be detected via an optical unit 162 in communication with the at least one detection chamber 120 of the one or more detection chambers 120. For example, during amplification, the plurality of nucleic acids of interest may be tagged, for example, using fluorophores. The optical unit 162 may detect an amount of fluorophores, which may be indicative of a presence, absence, and/or amount of the nucleic acid of interest in the biological sample.

FIG. 4 depicts an example diagnostic system, in accordance with present implementations. As illustrated by way of example in FIG. 4, an example diagnostic system 400 can include a cartridge and an instrument. The cartridge may be the same as or similar to the cartridge 100. Further, the instrument may be the same as or similar to the instrument 150.

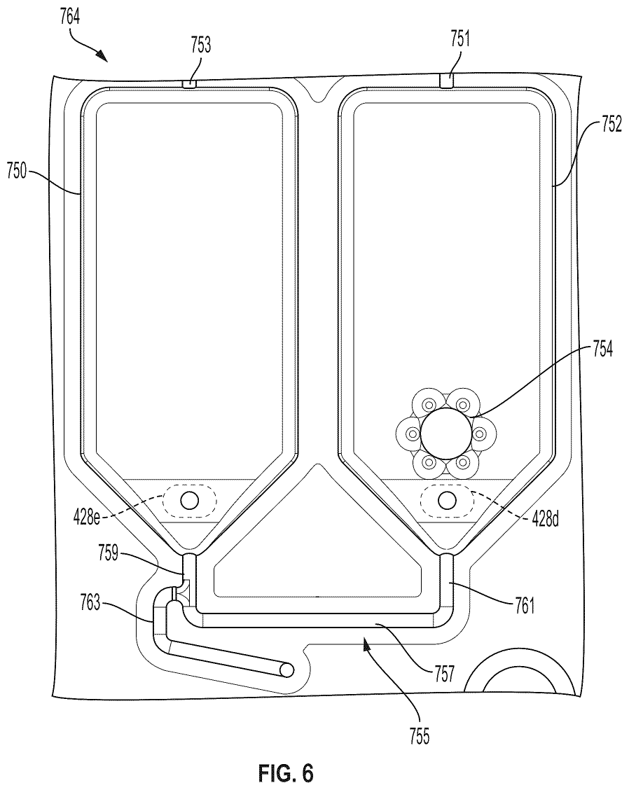

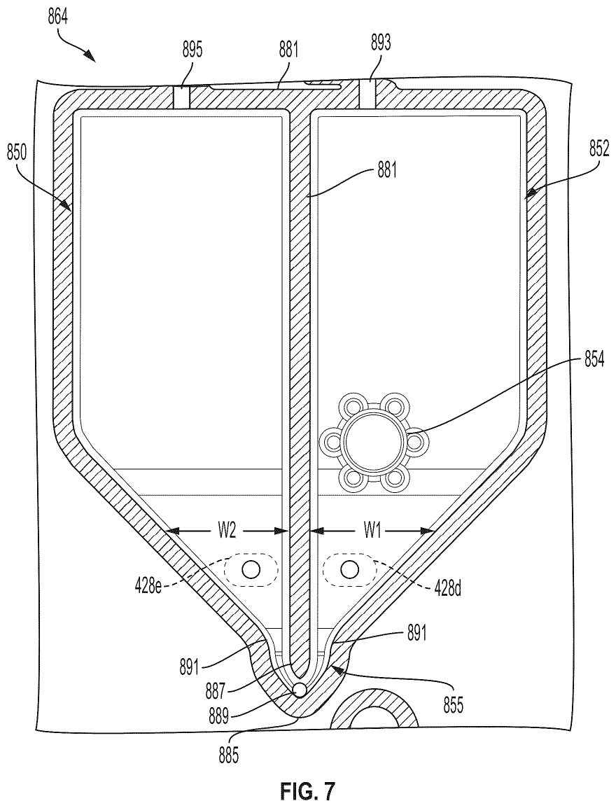

The cartridge of the example diagnostic system 400 may include a pump interface 404, a plurality of valves 408a-s, a plurality of frangible seals 410a-h, a liquid sample input chamber 412, a sample filter 414, a p-trap 416, an umbrella valve 418, a plurality of bubble traps 420a-d, a system composition blister devices 422, wash buffer blister devices 424a and 424b, a plurality of junctions 426a-d, a sample input (SI) metered section 430, an IPC 431, a lysis chamber 432 (e.g., the lysis chamber 114A) including lysing beads 436 and a filter for lysing 438, a PCA buffer 440 (stored in a blister device), a PCA buffer metered section 442, a master mix mixing chamber 444 having a master mix 446 (e.g., a lyophilized PC master mix), a vent membrane 448, a fluid mixing device 464 (e.g., similar to the fluid mixing device 114b), and a PCA chamber 456. The fluid mixing device 464 includes a first chamber 452 (e.g., similar to the chamber 115 of the fluid mixing device 114b) including a pellet of functionalized paramagnetic particles 454 (e.g., MB lyo particles, similar or the same as the magnetic particles 116) and a second chamber 450 (e.g., similar to the chamber 117 of the fluid mixing device 114b) fluidly coupled by a mixing channel 455. As discussed below, in some examples, the fluid mixing device 464 shown in FIG. 4 may be replaced by the fluid mixing device 114b shown in FIG. 1A, the fluid mixing device 764 shown in FIG. 6, or the fluid mixing device 864 shown in FIG. 7. In particular, the fluid mixing device 114b shown in FIG. 1 and the fluid mixing device 864 shown in FIG. 7 may provide faster homogenization of the mixture of lysate and functionalized paramagnetic particles, may improve fluid draining, and may substantially eliminate the formation of bubbles in the mixture compared to the fluid mixing device 764 of FIG. 6.

As shown in FIG. 4, the instrument of the diagnostic system 400 may include a pump 402 (e.g., air cylinder pumps, diaphragm pumps, a syringe pump (as shown), etc.), a plurality of fluid sensors 428a-f, an external magnet 458, and a an external sonicator with an ultrasonic horn 434. The elements of the instrument may be positioned such that certain components interface with certain components of the cartridge. For example, FIG. 4 indicates locations of fluid sensors 428a-f within the instrument relative to the elements within the cartridge. For example, fluid sensors 428a-f may be disposed within the instrument at various locations corresponding to elements located in the cartridge such that the sensors 428a-f sense fluid motion, movement, etc. within the cartridge at the indicated positions. For example, sensor 428a is shown to be located at an inlet of the SI metered section 430. The sensor 428a may not be physically located within the cartridge at the inlet of the SI metered section. Rather, the sensor 428a may be positioned within the instrument such that, upon insertion of the cartridge into the instrument, the position of the sensor 428a aligns with the inlet of the SI metered section 430. Further, as stated, the instrument of the diagnostic system 400 may also include a magnet 458 to interface with the PCA chamber 456. The magnet may be positioned within the instrument such that, upon insertion of the cartridge into the instrument, the location of the magnet 458 aligns with the position of the PCA chamber 456 within the cartridge. As discussed above the instrument of the diagnostic system 400 may also include a sonicator (e.g., similar or the same as the sonicator 154) including an ultrasonic horn 434. When the cartridge is inserted into the instrument, the ultrasonic horn 434 may align with the position of the lysis chamber 432 within the cartridge. Thus, when the sonicator is activated, the ultrasonic horn 434 may transmit ultrasonic waves into the lysis chamber 432 to lyse a sample therein.

In various examples, the system composition blister devices 422 and the wash buffer blister devices 424 may be similar to the blister devices 126a and 126b, respectively. The system composition stored in the blister device 422 may be similar to the system composition 106 and the wash buffer composition stored in the blister devices 424a and 424b may be similar to the wash buffer 110. The lysis chamber 432 may be similar to the lysis chamber 114a and the first and second chambers 450, 452 of the fluid mixing device 464 may be similar to the chambers 115, 117 of the fluid mixing device 114b. The PCA chamber 456 may also be similar to the detection chamber 120.

A user may fill the liquid sample input chamber 412 with a sample liquid. The sample liquid may be a biological sample containing one or more nucleic acids or other molecules of interest. The sample input chamber 412 may be similar to or the same as the sample input chamber 102. The user may fill the sample input chamber 412 via a pipette, an exact volume pipette, a dropper, syringe injection, etc. The sample input chamber 412 may include fill guides to indicate a fill level. Upon filling the sample input chamber 412, all 408a-s may be open. An umbrella valve 418 may disable the sample input chamber 412 from filling the sample metering circuit (e.g., the SI metered section 430). In various examples, the umbrella valve 418 may be a type of check valve. For example, the umbrella valve 418 may prevent flow back towards the liquid sample input chamber 412. The umbrella valve 418 may have a sufficient cracking pressure in a forward direction, thereby preventing the fluid in the sample input chamber 412 from reaching the sample metering section 430 from gravity (e.g., a head height pressure). Upon filling the liquid sample input chamber 412, the chamber may be closed. For example, a sample input cover of the cartridge may be closed.

Upon filling the sample input chamber 412, the sample may be pressurized by the pump 402 through valve 408a. Pressure may be vented through normally open valves 408k, 408n and/or 408o and the vent membrane 448. For example, pressure may be vented through valve 408k and vent channel 445. Thus, the sample may be pushed through the umbrella valve 418, the bubble trap 420a, and into the sample metering channels (e.g., the SI metered section 430). Fluid flow may be monitored as the liquid moves past an inlet of the SI metered section 430. In various examples, a first fluid flow sensor 428a may be positioned within the instrument such that the sensor aligns with and senses fluid at an inlet of the SI metered section 430. It should be understood that the positions of the fluid flow sensors 428 may be positioned to correspond to any locations of the cartridge 100. For example, a second fluid flow sensor 428b may also be positioned within the instrument such that the sensor 428b aligns with and senses fluid at an outlet of the SI metered section 430. The sensors 428a and 428b may monitor the fluid flow until the liquid reaches the sample metering outlet sensor 428b. Responsive to the liquid reaching the outlet sensor 428b, flow may be stopped. For example, valves may close to prevent movement of the liquid.

The fluid flow sensors may detect if a cartridge channel or chamber has liquid or air present. The fluid flow sensors may track progress of liquid slugs, meter liquid by triggering the halt of flow to control volume of fluid (e.g., sensors 428b, 428c, and 428f), help in reciprocating mixing by triggering when a flow should be reversed (e.g., sensors 428d, 428e, and 428f), and/or help locate the reconstituted master mix reagent in the PCA chamber (e.g., sensors 428c and 428f).

In various examples, sensors from the instrument 150 may be optical and/or capacitive. Optical sensors may be or include LEDs and photodiodes to detect changes in contrast, color, reflection, etc. in a microfluidic channel or chamber. Capacitive sensors may be or include capacitor plates to detect change in dielectric between liquid and air in a microfluidic channel or chamber.

Responsive to metering the sample, frangible seals 410a and 410b may be opened. Opening the frangible seals 410a and 410b may enable fluid flow into and out of the system composition blister device 422. The pump 402 may interface with the pump interface 404, which is fluidly coupled to the fluidic channels of the cartridge 100 and fluidly couplable to the pump. The cartridge (e.g., at least a portion of the fluidic channels in the cartridge) may be pressurized by the pump 402 through the pump interface 404 and pinch valve 408b, thus purging the sample from the SI metered section 430. Further, the sample may reconstitute an IPC lyo particle 431, and the sample may fill the lysis chamber 432. Pressure may be vented through one or more of pinch valves 408n, 408o, and/or 408k and/or the vent membrane 448 to allow the fluid flow. For example, pressure may be vented through the pinch valve 408k.

In various examples, the system composition fluid (e.g., system composition 106) may flow through the SI metered section 430 as the blister device 422 empties, pushing the sample out of the SI metered section 430. In various examples, it may be beneficial to fully empty the blister device 422 for improved performance. Air may then be pushed through the blister device 422 (e.g., via syringe pump 402) to fully empty the blister device 422 and purge any remaining system composition fluid from the SI metered section 430 into the lysis chamber 432. Metering sensors (e.g., sensors 428a and 428b) may manage a flow rate through the SI metered section 430 and minimize an amount of air pushed into the lysis chamber 432 from the system composition blister device 422.

Responsive to emptying the system composition blister device 422, the cartridge 100 may be pressurized through the pump interface 404 and the pinch valve 408d. The lysis chamber 432 may then be pressurized to ensure contact between a lysis chamber film and a lysis chamber heater. In various examples, all valves may be closed to isolate the lysis chamber 432 during lysis. In various examples, lysis may be an ultrasonic lysis. As such, the ultrasonic horn 434 may be pulsed, and a temperature of the lysis chamber 432 may be controller via the horn 434, the heater, and/or a temperature sensor. The ultrasonic pulses may lyse the fluid and mix the sample, the system composition, and the IPC lyo particle 431.

Responsive to lysing the sample, the cartridge 100 may be pressurized by the pump 402 through the pump interface 404, the pinch valve 408d, and the upstream channel 443. Further the valves 408p, 408o and/or 408n may be opened. This may allow the lysate (e.g., the lysed sample, the system composition, and the IPC lyo material mixture) to be delivered from the lysis chamber 432, out of the lysis chamber outlet 441, through a PCA common line 439 and the PCA chamber 456 into the first chamber 452 (e.g., a first hybridization chamber) or the second chamber 450 (e.g., a second hybridization chamber) of the fluid mixing device 464. The entrance of the lysate into the first chamber 452 or the second chamber 450 of the fluid mixing device 464 may cause the MB lyo particles 454 to be reconstituted. In various examples, all of the lysate is delivered from the lysis chamber 432. Flow may be stopped responsive to a determination that a trailing meniscus of the lysate is detected by a PCA inlet sensor (e.g., the fluid sensor 428c). The PCA chamber 456 may be kept full of fluid to minimize air generation in the chamber, which may disrupt amplification.

Responsive to the lysate entering the first chamber 452, hybridization may occur. For example, the lysate may be pumped back and forth between the first chamber 452 and the second chamber 450 of the fluid mixing device 464. The lysate may be pumped back and forth by alternating pressurization through the valve 408h (with venting through the valve 408n) and the valve 408g (with venting through the valve 408o). In order to minimize air generation, the chamber liquid sensors (e.g., sensors 428d and 428e) may monitor fluid flow to ensure neither the first chamber 452 nor the second chamber 450 is fully drained. Flow between the first chamber 452 and the second chamber 450 may allow mixing of the MB lyo particles 454 into the lysate. A heating rate of the fluid may be increased by mixing the fluid with itself. Mixing the fluid with itself may improve a heating rate because the heat source may be applied on one side of the liquid volume, reducing an amount of time to conduct the heat compared to when the liquid is in a static state. The region may include controlled heating during hybridization to promote capture of the sample target RNA to the paramagnetic particles.

After hybridization and mixing of the lysate, the lysate may be moved back toward the lysis chamber 432. For example, the lysate may move through the PCA chamber 456. A magnet 458 may interface with the PCA chamber 456 to pull the paramagnetic particles to a PCA foil (e.g., a heating element 122), thus separating the particles from the lysate. This may be referred to as a solid phase extraction. Flow of the lysate back to the lysis chamber 432 may occur by pressurizing through opened valves 408g and 408h and opening the valves 408p and 408k to allow flow to the lysis chamber 432. This process may ensure balanced draining of both the first chamber 450 and the chamber 452 of the fluid mixing device 464. In various examples, the PCA inlet sensor 428c may detect when all of the lysate has been purged from the PCA chamber 456.

In various examples, a plurality of wash buffers may be enabled for use in the cartridge 100. For example, two wash buffers may be used. In various examples, the first wash buffer may be a more aggressive chemistry that may be more inhibitory to a final PCA reaction relative to the second wash buffer. For example, the first wash buffer may include water, 0.017M of MgCl2, 0.11M of Tris-HCL for a pH of 8.0, and 0.056% of Tween® 20. In various examples, NaCl or KCl may be utilized rather than MgCl2.The first wash buffer may be enabled by opening the frangible seals 410c and/or 410d of the wash buffer blister device 424a. The first wash buffer may then be moved from the blister device 424a through the PCA chamber 456 by pressurizing through the valves 408c and 408i. Valves 408q, 408n, and 408o may be open to allow fluid flow. The volume of the first wash buffer may be controlled using the sensors 428d and 428e positioned within the instrument such that the sensor aligns with and senses fluid in the hybridization chambers. In various examples, the blister device 424a may not be fully emptied in order to minimize air passing downstream through a common channel to the PCA chamber 456. After passing a controlled volume of the first wash buffer through the PCA chamber 456, flow may be reversed by pressurizing valve 408g and 408h and opening valves 408q, 408i, and 408j to allow fluid flow. The first wash buffer may be returned to the blister device 424a until the end of the fluid flow is detected (e.g., by any sensor 428). Responsive to the detection of the end of the fluid flow, additional air and/or liquid may be purged to the lysis chamber 432.

In various examples, the second wash buffer may be a less aggressive chemistry that may be less inhibitory to a final PCA reaction relative to the first wash buffer. For example, the second wash buffer may include 0.01 M Tris buffer composition, 0.1 M NaCl, and 0.0015 M KCl, with a pH 8.0 at 25 degrees Celsius and 0.01% Tween® 20 when dissolved in one liter of deionized water. In some cases, the second wash buffer may include the same or a similar composition to the first wash buffer (e.g., water, 0.017M of MgCl2, 0.11M of Tris-HCL for a pH of 8.0, and 0.056% of Tween® 20). The second wash buffer may be enabled by opening the frangible seals 410e and 410f of the wash buffer blister device 424b. The second wash buffer may then be moved from the blister device 424b through the PCA chamber 456 by pressurizing through the valve 408c. Valves 408r, 408n, and 408o may be open to allow fluid flow. The volume of the second wash buffer may be controlled using the sensors 428d and 428e positioned within the instrument such that the sensors align with and sense fluid in the hybridization chambers. In various examples, the blister device 424b may not be fully emptied in order to minimize air passing downstream through a common channel to the PCA chamber 456. After passing a controlled volume of the second wash buffer through the PCA chamber 456, flow may be reversed by pressurizing through the opened valves 408g and 408h and opening valves 408r and 408j to allow fluid flow. The second wash buffer may be returned to the blister device 424b until the end of the fluid flow is detected (e.g., by any sensor 428). Responsive to the detection of the end of the fluid flow, additional air and/or liquid may be purged to the lysis chamber 432.

In various examples, the PCA buffer 440 may be metered (e.g., at the PCA buffer metered section 442). The PCA buffer blister valves 410g and 410h may be opened. Flow may then move out of the PCA buffer 440. Flow may be moved out by sucking flow at the valve 408f and opening the valves 408e and 408l to allow flow. A bubble trap 420d may be used to catch air from the PCA buffer 440 that moves downstream. The PCA buffer 440 may continue to fill the PCA buffer metered section 442 until the PCA buffer 440 is sensed by the PCA buffer metering sensor 428f.

After the PCA buffer flows past a location aligned with the sensor 428f, the valve 408e is closed and the valve 408m is opened to allow pressurized air to separate remaining PCA buffer from a controlled metered PCA buffer volume. The metered volume may be sucked into the master mix (MM) mixing chamber 444, and the master mix lyo bead may be reconstituted. Flow may be stopped responsive to a trailing meniscus of the PCA buffer is sensed by the PCA buffer metering sensor (e.g., the sensor 428f).

The master mix (MM) lyo material may be mixed well into the PCA buffer. The MM lyo material may be mixed into the PCA buffer by recirculating flow between the MM metering section and the MM mixing chamber 444. Flow may be recirculated by alternating positive and negative pump pressure with valve 408f and valve 408m open. Once mixing is complete, the master mix is pulled into the metering channels and out of the mixing chamber 444.

Responsive to the master mix being fully mixed, the master mix may be loaded into the PCA chamber 456. The master mix may enter the PCA chamber 456 by applying a vacuum to valve 408h, with valves 408s and 408m open to allow fluid flow. The master mix may enter the PCA well until a trailing liquidmeniscus of the master mix arrives at a location corresponding to the PCA inlet sensor 428c.

Responsive to the master mix arriving at the PCA chamber 456, PCA may be performed. The PCA chamber 456 may be pressurized via the valve 408h to reduce an impact of air bubble growth during PCA temperature cycling. After pressurization, the chamber may be isolated from the pump by closing all valves of the cartridge 100. An isothermal temperature in the PCA chamber 456 may be controlled via a plurality of heaters on each side of the PCA chamber 456. In various examples, electrical pulsing of the PCA foil (e.g., the heating element 122) may create temperature pulses for the PCA reaction.

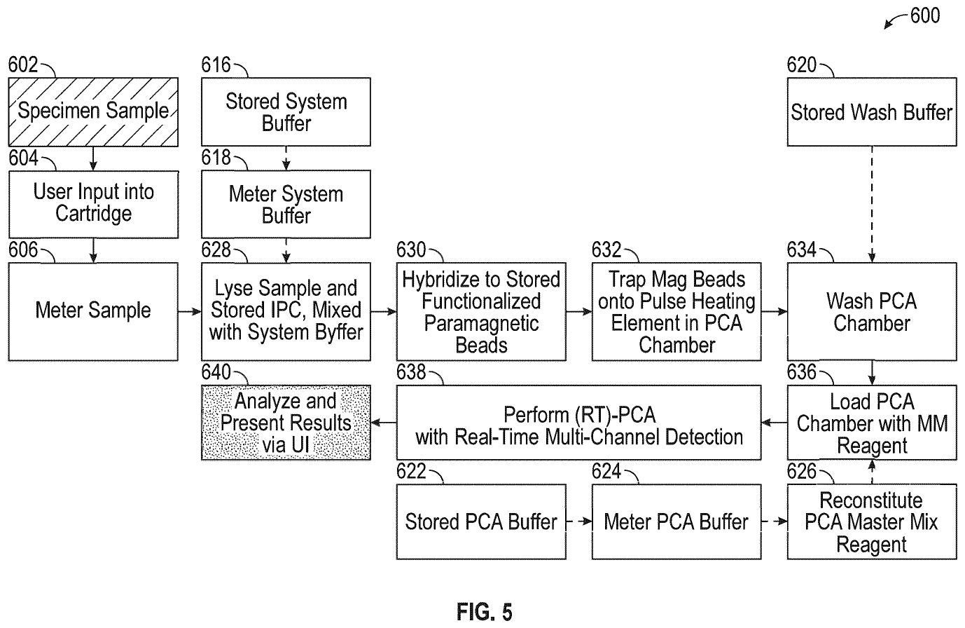

Referring now to FIG. 5, a method 600 for detecting the presence, absence, amount, etc. of a nucleic acid of interest, is shown according to some embodiments. Generally, a user may input an amount of sample (e.g., such as swab specimen eluted in a transport media) into the cartridge 100. The cartridge 100 may meter a proper amount of the sample and system composition 106, and internal positive control (IPC) may be added. The target organisms in the sample and IPC are lysed. Nucleic acids from target organisms and IPC may hybridize (e.g., bind) to paramagnetic particles. Paramagnetic particles with bound capture oligonucleotides may be captured onto a pulse heater in the detection chamber 120 with a magnetic field. The detection chamber 120 and captured magnetic particles 116 are washed with stored wash buffer 110. PCA buffer 440 may be metered and used to reconstitute Master Mix reagent used for PCA. Reconstituted Master Mix reagent may be loaded into the detection chamber 120 with trapped magnetic particles. PCA (e.g., RT-PCA) is performed with real-time multi-channel detection. Results of PCA and detection of target nucleic acids are reported to the user.

At block 602, a specimen sample may be collected. The specimen sample may be a biological sample collected from, for example, a human that contains a molecule of interest. For example, the biological sample may include one or more nucleic acids of interest. A molecule of interest may be a molecule to be amplified and detected. The molecule of interest may be used for various purposes, such as diagnosing the person that the specimen sample belongs to. In various examples, the specimen sample may be collected by a nasal swab or other retrieval device. Further, the specimen sample may be collected from the person and eluted in a commercial transport medium, such as Copan UTM®, to be input into the cartridge 100. At block 604, the sample may be input into the cartridge 100. For example, the sample may be input into the sample input chamber 102. The sample volume of the specimen may be greater than a predefined value (e.g., between 275 and 325 microliters). For example, the sample volume may be greater than 300 microliters. At block 606, the volume of the specimen inserted into the cartridge may be metered so that the sample volume used in the detection process is at or around the predefined value. For example, 320 microliters of the specimen may be collected, and the volume may be metered to 300 microliters to be inserted into the cartridge. Metering a sample may include, for example, utilizing the sample as stored in the cartridge 100 or as delivered by a user, a defined volume between a liquid sensor and a cut-off junction, and/or a defined volume between an overflow valve and a cut-off junction.

At block 616, the system composition (e.g., system composition 106) may be stored. For example, the system composition 106 may be stored in a blister device attached to the cartridge and/or a sealed chamber integrated into the cartridge body. The system composition 106 may be used for lysis and/or hybridization. For example, the system composition 106 may be stored in a blister device and transported to one or more extraction chambers 114 (e.g., one or both of the extraction chamber 114a and the hybridization chambers of the fluid mixing device 114b) for use in lysing the biological sample and/or hybridizing the nucleic acids of interest of the lysed biological sample.

In various examples, the system composition 106 may comprise water, salt, a buffering compound, and/or surfactants. The system composition 106 may include relatively high concentrations of salts. Types of salts found in the system composition 106 may include, for example, KCl, MgCl2 and/or NaCl. In various examples, the surfactant may be Tween® 20. Further, the cartridge 100 may contain a certain volume of the system composition 106. For example, the cartridge 100 may include between 300 and 800 microliters of the system composition 106. For example, the cartridge 100 may include 500 microliters of the system composition 106. At block 618, the volume of the system composition 106 may be metered so that the system composition volume used in the lysing and/or hybridization processes is at or around the predefined value. For example, 465 microliters of the specimen may be collected, and the volume may be metered to 450 microliters to be transported to and/or used in the extraction chamber 114a and/or the fluid mixing device 114b.

At block 620, the wash buffer (e.g., wash buffer 110) may be stored. For example, the wash buffer 110 may be stored in a blister device attached to the cartridge and/or a sealed chamber integrated into the cartridge body. The wash buffer 110 may be used for washing magnetic particles (e.g., magnetic particles 116) and/or the detection chamber 120. For example, the wash buffer 110 may be stored in a blister device and transported to one or more detection chambers 120 (e.g., a PCA chamber) for use in washing the detection chamber to prepare for amplification and detection of the nucleic acids of interest of the biological sample.

In various examples, the wash buffer 110 may comprise water, salt, a buffering compound, and/or surfactants. The wash buffer 110 may include moderately high concentrations of salts (e.g., relative to the salt concentrations in the system composition 106). Types of salts found in the wash buffer 110 may include, for example MgCl2 and/or NaCl. In various examples, the surfactant may be Tween® 20. The wash buffer 110 may also include KCl. Further, the cartridge 100 may contain a certain volume of the wash buffer 110. For example, the cartridge 100 may include between 100 and 400 microliters of the wash buffer 110. For example, the cartridge 100 may include 250 microliters of the wash buffer 110. In various examples, the cartridge 100 may include a plurality of wash buffers 110, each having a different composition and/or different uses. For example, a first wash buffer may include a stronger washing agent, and a second wash buffer may be milder. For example, the first wash buffer 110 may include 0.05 M tris buffer, 0.15 M NaCl, 0.0025 M KCl, and 0.05% Tween® 20, while the second wash buffer 110 may include 0.025 M tris buffer, 0.05 M NaCl, 0.0015 M KCl, and 0.01% Tween® 20. The first wash buffer may be used to wash the detection chamber 120, and the second wash buffer may be used to rinse the detection chamber 120.

At block 622, the PCA buffer (e.g., PCA buffer 440) may be stored. For example, the PCA buffer 440 may be stored in a blister device attached to the cartridge and/or a sealed chamber integrated into the cartridge body. The PCA buffer 440 may be used to reconstitute a master mix reagent used for reverse transcription and PCA amplification (e.g., the amplification process). For example, the PCA buffer 440 may be stored in a blister device and transported to one or more detection chambers 120 (e.g., a PCA chamber) for use preparing for amplification of the nucleic acids of interest.

In various examples, the PCA buffer 440 may comprise water and/or salt. The PCA buffer 440 may include small concentrations of salts (e.g., relative to the salt concentrations in the system composition 106 and/or the wash buffer 110). Types of salts found in the wash buffer 110 may include, for example MgCl2. In various examples, the PCA buffer 440 may include a surfactant (e.g., Tween® 20) and/or a buffering compound. Further, the cartridge 100 may contain a certain volume of the PCA buffer 440. For example, the cartridge 100 may include between 100 and 400 microliters of the PCA buffer 440. For example, the cartridge 100 may include 200 microliters of the PCA buffer 440.

At block 624, the volume of the PCA buffer 440 may be metered so that the PCA buffer volume used in the reconstitution process is at or around the predefined value. For example, 235 microliters of the specimen may be collected, and the volume may be metered to 60 microliters to be transported to and/or used in the detection chamber 120 Metering the PCA buffer 440 may control a concentration of the subsequently reconstituted master mix reagent, which may ensure proper amplification of the nucleic acids of interest. In various examples, the PCA buffer 440 may be metered by pumping the PCA buffer 440 through a defined volume between a liquid sensor and a fluidic T-junction bisecting the PCA buffer. Air may then be pumped into the T-junction such that a defined volume of the PCA buffer 440 is further transported.

At block 626, a PCA master mix reagent may be reconstituted. A master mix may be used for reverse transcription, PCA amplification, and/or real-time fluorescence detection. The master mix may be lyophilized (e.g., freeze dried). The PCA master mix reagent may be stored, for example, in a lyophilized pellet or cake, an air-dried pellet or cake, and/or sealed with a plastic plug or film.

In various examples, the PCA master mix reagent may include a plurality of active ingredients, such as: reverse transcriptase, polymerase, and dNTPs. For each reaction target, the master mix reagent may include a set of primers (e.g., one half of a primer pair used in PCR) and a fluorescent probe. In various examples, lyophilization excipients may include sugars, such as, for example, trehalose.

At block 628, the biological sample may be lysed, stored with internal positive control (IPC), and mixed with system composition. For example, the metered biological sample may be transported to the extraction chamber 114a. The system composition 106 and the IPC may be added to the extraction chamber 114a prior to, concurrent with, and/or subsequent to addition of the biological sample to the extraction chamber 114a. The IPC may be a lyophilized organism or synthetic organism. In various examples, the IPC may be a protein and/or organism present in a human sample. At block 628, the sample, the system composition, and the IPC may be mixed using sonication. For example, the lysis system 152 may be coupled with the extraction chamber 114a. The sonicator 154 may perform sonication to mix the sample, buffer, and IPC.

In various examples, the extraction chamber 114a may be heated by an external heater and/or the sonicator 154. Sonication may induce cavitation upon organisms (e.g., the IPC). In various examples, small beads or particles (e.g., ceramic beads, glass beads) may be included in the extraction chamber 114a. The beads or particles may be agitated to mechanically lyse the solution. In various examples, lysis may result from one or more of heat, cavitation, and/or the lysing beads.

At block 630, potential targeted nucleic acids in the solution lysed at block 628 may be hybridized to stored functionalized paramagnetic particles (e.g., magnetic particles 116). The solution lysed in the extraction chamber 114a may be referred to as a “lysate.” The lysate may be transported to the fluid mixing device 114b. In the fluid mixing device 114b, the lysate may reconstitute lyophilized paramagnetic particles. The lyophilized paramagnetic particles may be functionalized with capture oligonucleotides that may be designed to capture RNA and/or DNA strands from target organisms. The oligonucleotides may be utilized as a half of a primer pair during amplification (e.g., during the PCA amplification reaction).

During hybridization, the magnetic particles 116 may be mixed and agitated with the lysate. Mixing and agitation may optimize a capture efficiency of the target nucleic acids. In various examples, hybridization may occur between 55 and 65 degrees Celsius. For example, hybridization may occur at 62 degrees Celsius. In various examples, hybridization may occur with a high salt content (e.g., MgCl2 or NaCl). Mixing may occur, for example, by reciprocating flow between two chambers (e.g., a first hybridization chamber and a second hybridization chamber of the fluid mixing device 114b) such that vortices form at an entrance of each chamber to mix the lysate with the magnetic particles 116. For example, as discussed above with respect to FIG. 1A, the fluid mixing device 114b may include two chambers 115, 117, one or both of which may include magnetic particles 116 (e.g., lyophilized paramagnetic particles). By alternatingly pressurizing the chambers 115, 117, the lysate may be forced back and forth between the chambers 115, 117, causing the magnetic particles 116 to be mixed into the lysate.

At block 632, the magnetic particles 116 are trapped onto a heating element (e.g., the heating element 122) in the detection chamber 120 (e.g., a PCA chamber). The heating element 122 may be or include, in various examples, a continuous, structured, or shaped metal foil, metal wires, a conductor and/or resistor layer deposited and/or plated, and/or backed by a heat spreader. In various examples, in order for the PCA process to occur, the paramagnetic particles with the captured nucleic acids may be concentrated at a thermocycling zone of the heating element. Thus, after hybridization, the lysate hybridized with the magnetic particles may be transported to the detection chamber 120, where the particles are “trapped” or otherwise attached to the heating element 122.

The lysate may be delivered to the detection chamber 120. An external permanent magnet (e.g., magnetic field generator 156) may create a magnetic field. The generated magnetic field may attract the paramagnetic particles onto a surface of the heating element 122. The lysate may then be disposed. For example, the lysate may be stored in a location of the cartridge (e.g., the blister device used to store the system composition 106). In various examples, the magnetic particles may be distributed uniformly across the heating element 122. Further, in various examples, a flow of the lysate may be continuous, in discrete steps, or any combination thereof.

At block 634, the detection chamber 120 is washed using the wash buffer (e.g., the wash buffer 110). Washing the detection chamber 120 may remove undesirable components in the detection chamber 120 and/or on the magnetic particles 116 that may interfere with amplification of the nucleic acids of interest. The wash buffer 110 may be pumped through the detection chamber 120 to wash the chamber. Upon washing, the wash buffer 110 may return to the blister device storing the wash buffer. In various examples, a volume, time, flow rate, and/or flow directionality may be adjusted based on needs of the amplification and/or assay.

At block 626, as stated above, the PCA master mix reagent is reconstituted. In various examples, a metered amount of the master mix may be used to reconstitute the master mix reagent. The master mix reagent may be lyophilized. The PCA buffer 440 may be mixed with the master mix by reciprocally pumping between a storage chamber storing the original master mix reagent and a channel leading to the chamber.

At block 636, the detection chamber 120 is loaded with the reconstituted master mix reagent. In order for amplification to be performed, the master mix reagent may be located in the detection chamber 120 where temperature incubation and/or thermocycling occurs. In various examples, air bubbles may interfere with optical detection of the nucleic acid of interest. Thus, the system may remove any air bubbles in the detection chamber 120 after the master mix reagent is loaded. Air bubbles may be managed, for example, by air bubble traps with stagnant chamber geometries, hydrophobic vent membranes over a channel or chamber, columns, posts, filters, elongated vertical chambers for buoyant bubble collection, etc. In various examples, air may be minimized to reduce movement when the solution is heated. Specifically, air on at least one send of the reaction solution may be minimized. For example, a valve may be positioned on one or more entrances and/or exits to the detection chamber 120 to remove air from the detection chamber 120.

At block 638, amplification is performed. In various examples, amplification may be or include a PCA reaction, as will be described in greater detail with respect to FIG. 9. Upon amplification, a real-time multi-channel detection may be performed to detect the nucleic acids of interest. For example, the optical unit 162 may detect an absence, presence, and/or amount of the nucleic acid of interest. In various examples, detection may be performed using optical fluorescence and/or electrochemical detection with functionalized surfaces.

At block 640, the results of the amplification may be analyzed and presented. For example, the results may be analyzed to determine a presence, absence amount, etc. of the nucleic acid of interest. The results may be displayed, for example, via a user interface.

As discussed above, the system is configured to hybridized potential targeted nucleic acids in the lysate to stored functionalized paramagnetic particles (e.g., magnetic particles 116), for example, in block 630 of the method 600. For example, in the cartridge of the example diagnostic system 400, the first chamber 452 of the fluid mixing device 464 may include a pellet of functionalized paramagnetic particles 454 stored therein. After the lysate is pumped into the first chamber 452, the pump may alternatingly pressurize the first chamber 452 and the second chamber 450 causing reciprocating flow of the lysate between the two chambers 450, 452 through mixing channel 455 and dispersing the functionalized paramagnetic particles 454 within the lysate. FIG. 6 illustrates a fluid mixing device 764, which may be similar to the fluid mixing device 464 and may perform similar functions. Thus, in some examples, the cartridge 100 of the diagnostic system 400 illustrated in FIG. 4 may include the fluid mixing device 764 rather than the fluid mixing device 464. The fluid mixing device 764 includes a first chamber 752 and a second chamber 750, corresponding to and performing similar functions as the first chamber 452 and the second chamber 450 of the fluid mixing device 464, respectively. Like the first chamber 452 of the fluid mixing device 464, the first chamber 752 of the fluid mixing device 764 may include a pellet of functionalized paramagnetic particles 754. The first chamber 752 is fluidly coupled to a first pump inlet channel 751, which may be selectively coupled to a pump interface (e.g., pump interface 404 as illustrated in FIG. 4) or a vent membrane (e.g., vent membrane 448 as illustrated in FIG. 4), for example, via valves (e.g., valves 408h, 408o as illustrated in FIG. 4). The second chamber 750 of the fluid mixing device 764 is fluidly coupled to a second pump inlet channel 753, which may be selectively coupled to the pump interface (e.g., pump interface 404 as illustrated in FIG. 4) or the vent membrane (e.g., vent membrane 448 as illustrated in FIG. 4), for example, via valves (e.g., valves 408h, 408n as illustrated in FIG. 4).

The fluid mixing device 764 further includes a mixing channel 755 fluidly coupling the first chamber 750 to the second chamber 752 (e.g., similar to the mixing channel 455 as illustrated in FIG. 4). The mixing channel 755 includes a horizontal portion 757 connecting two vertical portions 759, 761 connected to the second chamber 750 and the first chamber 752, respectively. The vertical portion 761 is also fluidly coupled to a supply channel 763, which may be fluidly coupled to a PCA chamber (e.g., PCA chamber 456 via channel 468 as illustrated in FIG. 4) and a lysis chamber (e.g., lysis chamber 432 as illustrated in FIG. 4). As discussed above with respect to blocks 628-632 of the method 600, a lysate containing a sample and buffer material may be supplied to the first chamber 752 or the second chamber 750 of the fluid mixing device 764 from the lysis chamber 432 (as illustrated in FIG. 4) via the supply channel 763. The lysate may partially fill one or both of the chambers 750, 752 and may fill the mixing channel 755, the supply channel 763, and the PCA chamber 456 (as illustrated in FIG. 4). Then, the lysate in the chambers 750, 752 and the mixing channel 755 may be alternatingly pumped between the first chamber 752 and the second chamber 750 of the fluid mixing device 764 by alternatingly pressurizing the chambers 750, 752 with the pump 402 (as illustrated in FIG. 4) via the pump inlet channels 751, 753. This may cause reciprocating flow of the lysate between the two chambers 750, 752 and dispersing of the functionalized paramagnetic particles 754 within the lysate. As shown in FIG. 6, the sensors 428d, 428e of the instrument 150 may be aligned respectively with the first chamber 752 and the second chamber 750 and may be used, for example, to detect fluid in the chambers 750, 752 and to trigger when the flow of fluid should be reversed. After the functionalized paramagnetic particles 754 have been adequately mixed into the lysate (e.g., after the mixture is homogenized), both of the chambers 750, 752 may be pressurized to force the lysate out of the fluid mixing device 764 through the supply channel 763 and through the PCA chamber 456 back to the lysis chamber 432.

The design of the fluid mixing device 764 is adequate to homogenize the functionalized paramagnetic particles 754 within the lysate. However, the design tends to result in a relatively large amount of fluid (e.g., about 8 microliters) remaining in the mixing channel 755 and the supply channel 763 after the rest of the lysate has been pumped back into the lysis chamber 432 (as illustrated in FIG. 4). It is preferrable that all or substantially all of the lysate returns to the lysis chamber 432, as lysate remaining in the mixing channel 755 or the supply channel 763 may contaminate the PCA chamber 456 (as illustrated in FIG. 4) after the wash process of block 634 of the method 600 of FIG. 5 Further, the target molecules bound to functionalized paramagnetic particles 754 in the left-behind fluid may also not reach the PCA chamber 456, which may reduce the sensitivity of the diagnostic test. The long length of the mixing channel 755 may result in trapped fluid menisci (or fluid slugs) with pockets of air therebetween. This can cause bubbles to form in the PCA chamber 456, may interfere with the downstream diagnostic processes. The long length of the mixing channel 755 may also result in increased fluid resistance, this may drive up the time it takes to pump lysate between the first chamber 752 and the second chamber 750 of the fluid mixing device 764. For example, it may take up to 15-20 seconds to pump fluid between the chambers 750, 752. Accordingly, it may be advantageous to provide a design that reduces residual fluid in the fluid mixing device 764 after mixing, reduces the occurrence of bubbles, and speeds up the mixing process.

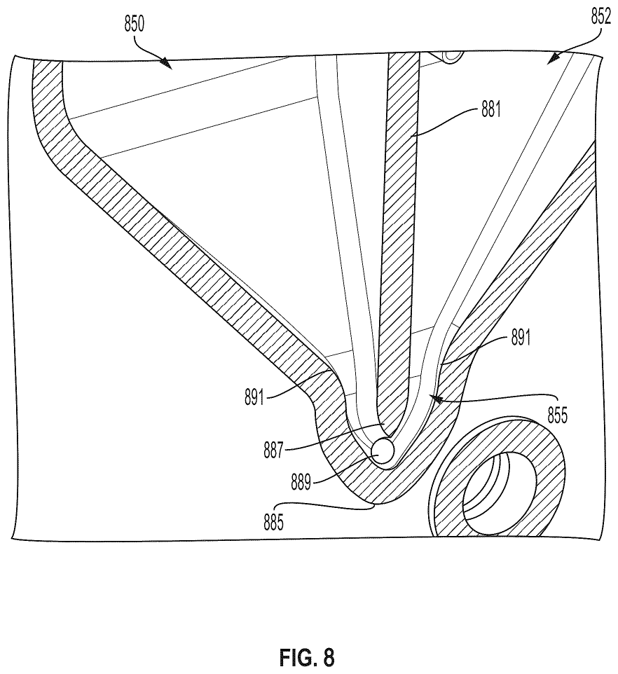

Referring now to FIGS. 8 and 9, a fluid mixing device 864 is shown according to an example embodiment. The fluid mixing device 864 may be similar to the fluid mixing device 464 and the fluid mixing device 764 and may perform similar functions. Thus, in some examples, the cartridge 100 of the diagnostic system 400 illustrated in FIG. 4 may include the fluid mixing device 864 rather than the fluid mixing device 464 or the fluid mixing device 764. The fluid mixing device 864 includes a first chamber 852 and a second chamber 850, corresponding to and performing similar functions as the first chamber 752 (and the first chamber 452) and the second chamber 750 (and the second chamber 450) of the fluid mixing device 764, respectively. The first chamber 852 of the fluid mixing device 864 may include a pellet of functionalized paramagnetic particles 854, similar to the pellet of functionalized paramagnetic particles 754 in the first chamber 752 of the fluid mixing device 764 (and the functionalized paramagnetic particles 454 in the first chamber 452 of the fluid mixing device 464). However, as can be seen in FIG. 7, the fluid mixing device 864 does not include a mixing channel with a horizontal portion similar to the horizontal portion 757 of the mixing channel 755 fluid mixing device 764 shown in FIG. 6. Instead, the fluid mixing device 864 includes a protrusion 881 separating the first chamber 852 from the second chamber 850 (which may be referred to as a first chamber and a second chamber). The protrusion 881 may extend from a top wall 883 of the fluid mixing device 864 to a first end 887 proximate but not reaching a bottom wall 885 of the fluid mixing device 864. The fluid mixing device 864 includes a U-shaped channel 855 that extends around the first end 887 of the protrusion 881 and fluidly couples the first chamber 852 of the fluid mixing device 864 to the second chamber 850 of the fluid mixing device 864. The first end 887 of the protrusion 881 may be at a bottom end of the protrusion, and the U-shaped channel 855 may be below the first chamber 852 and the second chamber 850. The U-shaped channel 855 transitions directly from a first portion extending upward toward the second chamber 850 to a second portion extending upward toward the first chamber 852 without a horizontal segment extending therebetween. In some examples, a bottom portion of the U-shaped channel 855 is continuously concave. Thus, the U-shaped channel 855 does not include a horizontal portion similar to the horizontal portion 757 of the fluid mixing device 764.

The U-shaped channel 855 includes or is fluidly coupled to an opening 889, which may be fluidly coupled to a PCA chamber (e.g., the PCA chamber 456) via a fluidic channel (e.g., channel 468). The fluidic channel may supply fluid to the fluid mixing device 864 for mixing and drain fluid from the fluid mixing device 864 after mixing. As shown in FIGS. 8 and 9, the opening 889 is located at the bottom of the U-shaped channel 855. It should be understood that the fluid mixing device 864 is shown in an orientation as it would be when the cartridge 100 is installed in the instrument 150. Thus, while the cartridge 100 may be positioned in any orientation outside of the instrument 150, the cartridge 100 and the fluid mixing device 864 may be considered to have a “top” and a “bottom” defined by the orientation in which the cartridge is installed in the instrument 150 and as shown in the Figures.

The first chamber 852 and the second chamber 850 of the fluid mixing device 864 each form a funnel-like shape to guide fluid toward the U-shaped channel 855. The first width W1 of the first chamber 852 and the second width W2 of the second chamber 850 both decrease as the chambers 850, 852 approach the U-shaped channel 855 (e.g., in the downward direction as shown in FIG. 7). For example, as shown in FIG. 7, the outer walls of the chambers 850, 852 are sloped toward the protrusion 881 as they approach the U-shaped channel 855. Similarly, a first depth (e.g., into the page as shown in FIG. 7) of the first chamber 852 and a second depth of the second chamber 850 both decrease as the chambers 850, 852 approach the U-shaped channel 855. The cartridge 100 may include a flat closure plate or film that is coupled (e.g., welded) to the edges of the chambers 850, 852 and the protrusion 881 to fluidly seal the cartridge. As shown in FIG. 7, the depths of the first chamber 852 and the second chamber 850 are greatest near the top of the fluid mixing device 864. Closer to the bottom of the fluid mixing device 864, the depths decrease as the rear walls of the chambers 850, 852 approach the closure plate or film coupled to the edges of the chambers 850, 852 before meeting the U-shaped channel 855. Thus, the chambers 850, 852 narrow both in width and depth as they approach the U-shaped channel 855.

Each side of the U-shaped channel 855 may similarly decrease in width and depth approaching the opening 889 from either side. In some embodiments, a cross section of the U-shaped channel 855 may be about 1 millimeter wide and about 1 millimeter deep. This cross-section may be, for example, a cross-section on a vertical plane extending through the protrusion 881 and into the page as shown in FIG. 7. As shown in FIG. 7, the rate of decrease of the width of the U-shaped channel 855 (e.g., a third width) is lower than the rates of decrease of the widths of the chambers 850, 852. For example, the inner walls of the fluid mixing device 864 include convex portions 891 where each chamber 850, 852 meets the U-shaped channel 855. Said another way, the walls of the fluid mixing device 864 do not taper in a straight line to the bottom of the U-shaped channel 855 but taper toward the first end 887 of the protrusion 881 before curving downward and around the first end first end 887 of the protrusion 881 to form the U-shaped channel 855. This may increase fluid velocity (e.g., improved fluid jetting) as lysate is pumped through the U-shaped channel 855 between the chambers 850, 852, which may improve mixing of lysate and the functionalized paramagnetic particles 854 as the high velocity lysate exiting the U-shaped channel 855 meets the fluid already in the receiving chamber 850, 852, causing eddies (swirling fluid, vortices, etc.). The same fluid velocity would not be achieved if the walls of the fluid mixing device 864 tapered in a straight line to the bottom of the U-shaped channel 855

As discussed above, the opening 889 may be positioned at the bottom of the U-shaped channel 855, for example, directly below the protrusion 881 and symmetrically positioned between the two sides of the U-shaped channel 855 and between (and below) the chambers 850, 852. The U-shaped channel 855 may narrow as it approaches the opening 889 to guide fluid toward the opening in order to reduce residual fluid after draining the chambers 850, 852. However, as discussed above, the width of the U-shaped channel 855 may narrow at a lower rate than the widths W1, W2 of the chambers 850, 852 narrow. For example, a third width of a first side of the U-shaped channel 855 (e.g., portion of the U-shaped channel 855 on the right side of the protrusion 881 as shown in FIG. 8) may decrease as the U-shaped channel 855 approaches the opening 889. However, the rate of decrease of width (e.g., per unit length along the fluid flow path) of the third width may be less than the rate of decrease of the first width W1 of the first chamber 852. Similarly, a third depth of the first side of the U-shaped channel 855 may decrease as the U-shaped channel 855 approaches the opening 889. However, the rate of decrease of depth (e.g., per unit length along the fluid flow path) of the third depth may be less than the rate of decrease of the first depth of the first chamber 852.

As discussed above, the chambers 850, 852 of the fluid mixing device 864 may alternatingly be pressurized to force the lysate into the opposite chamber 850, 852, mixing the lysate with the functionalized paramagnetic particles 854 due to the motion of the fluid. For example, as shown in FIG. 7, a top portion of the first chamber 852 includes a first upper opening 893, which may be selectively coupled to a pump interface (e.g., pump interface 404 as illustrated in FIG. 4) or a vent membrane (e.g., vent membrane 448), for example, via valves (e.g., valves 408h, 408o, respectively, as illustrated in FIG. 4)). A top portion of the second chamber 850 includes a second upper opening 895, which may be selectively coupled to the pump interface (e.g., pump interface 404) or the vent membrane (e.g., vent membrane 448 as illustrated in FIG. 4)), for example, via valves (e.g., valves 408g, 408n, respectively, as illustrated in FIG. 4)). For example, the valve 408h may be positionable to fluidly couple the first chamber 852 to the pump interface 404 via the first upper opening 893 using a first valve actuator, and valve 408g may be positionable to fluidly couple the second chamber 850 to the pump interface 404 via the second upper opening 895 using a second valve actuator. The valve actuators may be components of the instrument 150 and may be controlled by the controller 164. By pressurizing one of the chambers 850, 852 (e.g., using the pump 402 as illustrated in FIG. 4), lysate may be pushed into the opposite chamber 850, 852, encouraging mixing of lysates and functionalized paramagnetic particles 854.

FIG. 9 shows the fluid mixing device 864 in the process of mixing lysate with the functionalized paramagnetic particles 854. In particular, the fluid mixing device 864 is shown while the second chamber 850 is being pressurized to force the lysate into the first chamber 852. The shape of the U-shaped channel 855 may cause eddies 857 (swirling fluid, vortices, etc.) to form in the lysate in the first chamber 852 as the high velocity lysate leaving the U-shaped channel 855 mixes with the lysate already in the first chamber 852. The pellet of functionalized paramagnetic particles 854 may be aligned with a first outlet of the U-shaped channel 855, such that, when the second chamber 850 is pressurized, the eddies 857 formed in the mixture in the first chamber 852 may be immediately adjacent (e.g., in contact with) the pellet of functionalized paramagnetic particles 854. The swirling flow of fluid around the pellet may encourage and accelerate dispersal of the functionalized paramagnetic particles 854 into the lysate, accelerating the homogenization process. As used herein, “aligned” means that a straight line representing the average direction of flow of fluid leaving the U-shaped channel 855 (e.g., line 859) passes through the pellet of functionalized paramagnetic particles 854.

The shape and short length of the U-shaped channel 855 may reduce the fluidic resistance and increase the fluid velocity (e.g., compared to the mixing channel 755 of the fluid mixing device 764 of FIG. 6), which may allow the length of the mixing cycles to be shortened. For example, where the fluid mixing device 764 may require 15-20 second mixing cycles, the fluid mixing device 864 may use 5-8 second mixing cycles, which may reduce mixing time by up to 75 percent. The shape and short length of the U-shaped channel 855 may also reduce the overall footprint of the 864 compared to the fluid mixing device 764. Because both of the chambers 850, 852 of the fluid mixing device 864 funnel to a common point at the bottom of the U-shaped channel 855, the “dead space” above the horizontal portion 757 of the fluid mixing device 764 of FIG. 6 is eliminated. Thus, the fluid mixing device 864 may support mixing of volumes of up to 1200 microliters in a relatively small footprint in the cartridge.

As discussed above, the opening 889 may be positioned at the bottom of the U-shaped channel 855, for example, directly below the protrusion 881 and symmetrically positioned between the two sides of the U-shaped channel 855 and between (and below) the chambers 850, 852. The opening 889 may be fluidly coupled to a fluidic channel that fluidly couples the fluid mixing device 864 to a pulse-controlled amplification chamber (e.g., the PCA chamber 456 as illustrated in FIG. 4) and upstream to a lysis chamber (e.g., the lysis chamber 432 as illustrated in FIG. 4). Fluid (e.g., lysate) may be supplied to the fluid mixing device 864 via the fluid channel and the opening 889 (e.g., from the lysis chamber 432) for mixing and drained from the fluid mixing device 864 via the fluid channel 468 (as illustrated in FIG. 4) and the opening 889 after mixing. The opening 889 may extend through a rear wall of the fluid mixing device 864 to fluidly couple to the fluidic channel 468 (as illustrated in FIG. 4). Thus, in some examples, the opening 889 in the U-shaped channel 855 extends through a rear wall of the fluid mixing device 864 and is located at the bottom of the U-shaped channel 855. As discussed above, it may be preferrable that all or substantially all of the lysate returns to the lysis chamber 432 to reduce contamination of the PCA chamber 456. The shape of the U-shaped channel 855 and the position of the opening 889 at the bottom of the U-shaped channel 855 and centered between the chambers 850, 852 may result in all or nearly all of the mixture being forced out of the fluid mixing device 864 (e.g., with no more than 1 microliter remaining) when both chambers 850, 852 are pressurized after homogenization of the mixture of lysate and functionalized paramagnetic particles 854.

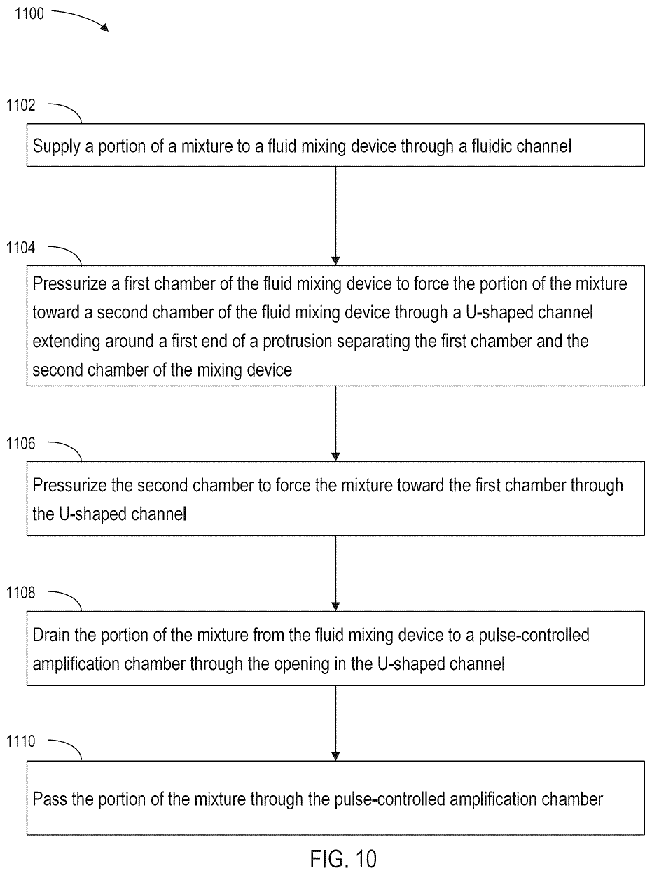

Referring now to FIG. 10, a method 1100 of homogenizing a mixture is shown according to an example embodiment. The method may be performed, for example, with the diagnostic system 400, with the fluid mixing device 864 of FIG. 7 replacing the fluid mixing device 464 shown in FIG. 4. At operation 1102 of the method 1100, a portion of a mixture is supplied to a mixing device through a fluidic channel (e.g., channel 468). The fluid mixing device (e.g., fluid mixing device 864) may include a first chamber (e.g., the first chamber 852), a second chamber (e.g., the second chamber 850), a protrusion separating the first chamber from the second chamber (e.g., protrusion 881), and a U-shaped channel (e.g., U-shaped channel 855) extending around a first end of the protrusion and fluidly coupling the first chamber to the second chamber. The U-shaped channel includes an opening (e.g., protrusion 881) fluidly coupled to the fluidic channel.

A mixture (e.g., a lysate) of a biological sample and a buffer fluid may be formed in the lysis chamber 432 (and cells in the biological sample may be lysed by the ultrasonic horn 434 (as illustrated in FIG. 4). The mixture may then be supplied to the first chamber 852 of the fluid mixing device 864 for example, by opening the pinch valve 408d, and valves 408p, 408o and using the pump 402 to pressurize the upstream channel 443 upstream of the lysis chamber 432 (as illustrated in FIG. 4). A portion of the mixture may remain in the channels and PCA chamber 456 between the lysis chamber 432 and the fluid mixing device 864. The upstream pressure may force the mixture into the first chamber 852. In some examples, the mixture may be supplied at operation 1102 to the second chamber 850 rather than the first chamber 852 (e.g., by opening valve 408n rather than valve 408o) or to both of the chambers 850, 852 (e.g., by opening both valves 408n, 408o). Thus, the second chamber 850 may be considered or referred to as the “first chamber” rather than the “second chamber” in the method 1100. As discussed above, the controller 164 (as illustrated in FIG. 2) may be used to operate the pump 402 (as illustrated in FIG. 4) and to operate valve actuators to selectively open and close the valves in the cartridge 100.

At operation 1104 of the method 1100, the first chamber is pressurized to force the mixture toward the second chamber through the U-shaped channel. For example, valve 408p may be closed to stop fluid from flowing back toward the lysis chamber 432, valve 408h may be opened to fluidly couple the first chamber 852 to the pump 402 via the first upper opening 893, and valve 408n may be opened to fluidly couple the second chamber 850 to the to the vent membrane 448 via the second upper opening 895. The pump 402 may pressurize the first chamber 852 to force the mixture (e.g., a mixture of lysate and functionalized paramagnetic particles 854) into the second chamber 850. In some examples, less than all of the fluid may be forced out of the first chamber, which may reduce the likelihood of bubbles forming in the mixture.

At operation 1106 of the method 1100, the second chamber is pressurized to force the mixture toward the first chamber through the U-shaped channel. Operation 1106 may thus be essentially the opposite of operation 1104. For example, with valve 408p remaining closed, valves 408h and 408n may be closed, valve 408g may be opened to fluidly couple the second chamber 850 to the pump 402 via the first upper opening 893, and valve 408o may be opened to fluidly couple the first chamber 852 to the to the vent membrane 448 via the second upper opening 895. The pump 402 may pressurize the second chamber 850 to force the mixture into the first chamber 852. In some examples, less than all of the fluid may be forced out of the second chamber, which may reduce the likelihood of bubbles forming in the mixture. As shown in FIG. 7, the sensors 428d, 428e of the instrument 150 may be aligned respectively with the first chamber 852 and the second chamber 850 and may be used, for example, to detect fluid in the chambers 850, 852 and to trigger when the flow of fluid should be reversed. As discussed above, the first chamber (e.g., the first chamber 852) may include lyophilized magnetic beads (e.g., functionalized paramagnetic particles 854), which may, for example, be formed into a pellet and coupled to a wall of the first chamber. Pressurizing the first chamber (e.g., the first chamber 852) to force the mixture toward the second chamber (e.g., the second chamber 850) and pressurizing the second chamber to force the mixture toward the first chamber through the U-shaped channel may cause the lyophilized magnetic beads to be dispersed within the mixture. The pellet of lyophilized magnetic beads may be aligned with a first outlet of the U-shaped channel such that pressurizing the second chamber to force the mixture toward the first chamber through the U-shaped channel causes an eddy to form in the mixture immediately adjacent (e.g., in contact with) the pellet. Operations 1104 and 1106 may be repeated as necessary to mix or homogenize the functionalized paramagnetic particles 854 in the lysate.

At operation 1108 of the method 1100, the portion of the mixture is drained from the fluid mixing device to a pulse-controlled amplification chamber through the opening in the U-shaped channel. Draining the portion of the mixture from the fluid mixing device may include pressurizing the first chamber and the second chamber (e.g., simultaneously). For example, the valve 408p may be opened to allow fluid to flow back into the lysis chamber 432, and both of valves 408g, 408h may be opened to fluidly couple both chambers 850, 852 to the pump 402. The pump 402 may pressurize both chambers 850, 852 to force the portion of the mixture out of the chambers 850, 852 through the U-shaped channel 855 and the opening 889, into the channel 468. The channel 468 may convey the portion of the mixture to the PCA chamber 456. It should be understood that draining the portion of the mixture from the fluid mixing device may refer to draining less than all of the portion of the mixture. For example, as discussed above, up to about 1 microliter may remain in the fluid mixing device 864.

At operation 1110 of the method 1100, the portion of the mixture, after being drained from the fluid mixing device 864 in operation 1108, is passed through the pulse-controlled amplification chamber. For example, the portion of the mixture may pass through the PCA chamber 456 (as illustrated in FIG. 4) where at least some of the functionalized paramagnetic particles 854 may be captured by the magnet 458 while the fluid passes through to the lysis chamber 432. After completion of the method 1100, the remaining steps of the diagnostic process may be performed (e.g., operations 634-640 of the method 600 of FIG. 5).

Thus, as an example of the method 1100, a portion of a mixture or lysate (e.g., a mixture of sample and buffer fluid) may be supplied to the first chamber 852 from the lysis chamber 432 via channel 468 (and passing through the PCA chamber 456) as illustrated in FIG. 4. The mixture may also fill the PCA chamber 456 and channel 468 between valve 408p and the fluid mixing device 864. Some of the mixture may also be supplied to the second chamber 850. Then, the first chamber 852 may be pressurized to force the portion of the mixture toward the second chamber 850 through the U-shaped channel 855 until most of the portion of the mixture is in the second chamber 850. The second chamber 850 may then be pressurized to force the portion of the mixture toward the back toward the first chamber 852 through the U-shaped channel 855 until most of the portion of the mixture is in the first chamber 852. The first chamber 852 and the second chamber 850 may be alternatively pressurized repeatedly to mix the fluid with the functionalized paramagnetic particles 854 (lyophilized magnetic beads). Pressurizing the second chamber 850 may cause fluid exiting the U-shaped channel 855 to form eddies in the first chamber 852 adjacent the pellet of functionalized paramagnetic particles 854, further encouraging mixing. After the functionalized paramagnetic particles 854 are sufficiently mixed into the mixture (e.g., after the mixture is homogenized), the portion of the mixture may be drained from the fluid mixing device 864 to the PCA chamber 456 (as illustrated in FIG. 4) through the opening 889, by opening valve 408p and pressurizing both the first chamber 852 and the second chamber 850, and may be passed through the PCA chamber 456 such that the functionalized paramagnetic particles 854 are captured by the magnet 458.

In some embodiments, rather than alternatingly pressurizing the chambers of the fluid mixing device, the chambers may be alternatingly subjected to negative pressure (e.g., vacuum). For example, at operation 1104, rather than pressurizing the first chamber to force fluid into the second chamber, the second chamber may be subjected to negative pressure to pull fluid into the second chamber. Similarly, in operation 1106, rather than pressurizing the second chamber to force fluid into the first chamber, the first chamber may be subjected to negative pressure to pull fluid into the first chamber. To create negative pressure the pump may be operated in reverse. For example, in the diagnostic system 400 of FIG. 4, where the fluid mixing device 864 is included in place of the fluid mixing device 464, the pump 402 may be operated in reverse to create suction (negative pressure) in the cartridge 100. While the 402 is creating suction, valve 408g may be opened to fluidly couple the second chamber 850 to the pump interface 404, and valve 408o may be opened to fluidly couple the first chamber 852 to the vent membrane 448. This may create negative pressure in the second chamber 850, which may pull fluid into the second chamber 850. To move fluid back toward the first chamber 852, valve 408g and 408o may be closed, and valve 408h may be opened to fluidly couple the first chamber 852 to the pump interface 404, and valve 408n may be opened to fluidly couple the second chamber 850 to the vent membrane 448, creating negative pressure in the first chamber 852.

Negative pressure may similarly be used to drain the mixture from the fluid mixing device in operation 1108. For example, with valves 408g and 408h closed, valves 408n and 408o may be opened to fluidly couple the first upper opening 893 of the first chamber 852 and the second upper opening 895 of the second chamber 850 to the vent membrane 448. Valves 408p and 408d may be opened to fluidly couple the 889 of the fluid mixing device 864 to the pump interface 404. The pump 402 may be operated in reverse to generate negative pressure, which may pull the mixture through the 889 into the channel 468 toward the PCA chamber 456.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A,’ only ‘B,’ as well as both ‘A’ and ‘B.’ Such references used in conjunction with “comprising” or other open terminology can include additional items. References to “is” or “are” may be construed as nonlimiting to the implementation or action referenced in connection with that term. The terms “is” or “are” or any tense or derivative thereof, are interchangeable and synonymous with “can be” as used herein, unless stated otherwise herein.

Directional indicators depicted herein are example directions to facilitate understanding of the examples discussed herein and are not limited to the directional indicators depicted herein. Any directional indicator depicted herein can be modified to the reverse direction or can be modified to include both the depicted direction and a direction reverse to the depicted direction, unless stated otherwise herein. While operations are depicted in the drawings in a particular order, such operations are not required to be performed in the particular order shown or in sequential order, and all illustrated operations are not required to be performed. Actions described herein can be performed in a different order. Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any clam elements.

The various ranges provided herein include the stated range and any value or sub-range within the stated range. Furthermore, when “about” is utilized to describe a value or percentage this includes, refers to, and/or encompasses variations (up to +/−ten %) from the stated value or percentage. In describing and claiming the examples disclosed herein, the singular forms “a”, “an”, and “the” include plural referents unless the context clearly dictates otherwise.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. For example, the method 200 may include additional operations not explicitly recited or may exclude certain recited operations in some examples. Various examples of the cartridge 100 or the instrument 150 may include additional components not explicitly recited, may exclude certain recited components, or may include the recited components in different relative positions than shown in the examples described above. Therefore, it is intended that the scope of this disclosure be limited only by the claims and the equivalents thereof.

What is claimed is:

1. A diagnostic cartridge comprising:
a fluid mixing device comprising:
a first chamber;
a second chamber;
a protrusion separating the first chamber from the second chamber; and
a U-shaped channel extending around a first end of the protrusion and fluidly coupling the first chamber to the second chamber;
a pulse-controlled amplification chamber; and
a fluidic channel fluidly coupling the pulse-controlled amplification chamber to an opening in the U-shaped channel to supply fluid to the fluid mixing device for mixing and to drain the fluid from the fluid mixing device after mixing.

2. The diagnostic cartridge of claim 1, wherein a first width and a first depth of the first chamber both decrease as the first chamber approaches the U-shaped channel, and a second width and a second depth of the first chamber both decrease as the second chamber approaches the U-shaped channel.

3. The diagnostic cartridge of claim 1, wherein a third width of a first side of the U-shaped channel decreases as the U-shaped channel approaches the opening, wherein a rate of decrease of the third width of the first side of the U-shaped channel is lower than a rate of decrease of the first width.

4. The diagnostic cartridge of claim 1, further comprising lyophilized magnetic beads in the first chamber.

5. The diagnostic cartridge of claim 4, wherein the lyophilized magnetic beads are formed into a pellet aligned with a first outlet of the U-shaped channel.

6. The diagnostic cartridge of claim 1, wherein the opening in the U-shaped channel extends through a rear wall of the fluid mixing device and is located at the bottom of the U-shaped channel.

7. The diagnostic cartridge of claim 1, wherein a cross-section of the U-shaped channel is about 1 millimeter wide and about 1 millimeter deep.

8. The diagnostic cartridge of claim 1, wherein the U-shaped channel transitions from a first portion extending upward toward the first chamber to a second portion extending upward toward the second chamber without a horizontal segment extending therebetween.

9. A method of homogenizing a mixture, the method comprising:
supplying a portion of the mixture to a fluid mixing device through a fluidic channel, the fluid mixing device comprising:
a first chamber;
a second chamber;
a protrusion separating the first chamber from the second chamber; and
a U-shaped channel extending around a first end of the protrusion and fluidly coupling the first chamber to the second chamber, the U-shaped channel including an opening fluidly coupled to the fluidic channel;
pressurizing the first chamber to force the portion of the mixture toward the second chamber through the U-shaped channel;
pressurizing the second chamber to force the portion of the mixture toward the first chamber through the U-shaped channel;
draining the portion of the mixture from the fluid mixing device to a pulse-controlled amplification chamber through the opening in the U-shaped channel; and
passing the portion of the mixture through the pulse-controlled amplification chamber.

10. The method of claim 9, wherein draining the portion of the mixture from the fluid mixing device comprises pressurizing the first chamber and the second chamber.

11. The method of claim 9, wherein the fluid mixing device further comprises lyophilized magnetic beads in the first chamber, wherein pressurizing the first chamber to force the mixture toward the second chamber and pressurizing the second chamber to force the mixture toward the first chamber causes the lyophilized magnetic beads to be dispersed within the mixture.

12. The method of claim 11, wherein the lyophilized magnetic beads are formed into a pellet coupled to a wall of the first chamber and aligned with a first outlet of the U-shaped channel such that pressurizing the second chamber to force the mixture toward the first chamber causes an eddy to form in the mixture immediately adjacent the pellet.

13. A diagnostic cartridge comprising:
a pump interface fluidly coupleable to a pump;
a fluid mixing device comprising:
a first chamber;
a second chamber;
a protrusion separating the first chamber from the second chamber; and
a U-shaped channel extending around a first end of the protrusion and fluidly coupling the first chamber to the second chamber;
a fluidic channel fluidly coupled to a bottom portion of the U-shaped channel to supply fluid to and drain fluid from the fluid mixing device;
a pulse-controlled amplification chamber fluidly coupled to the fluidic channel to receive the fluid from the fluid mixing device;
a first opening at a top portion of the first chamber;
a second opening at a top portion of the second chamber;
a first valve positionable to fluidly couple the first opening at the top portion of the first chamber to the pump interface; and
a second valve positionable to fluidly couple the second opening at the top portion of the second chamber to the pump interface.

14. The diagnostic cartridge of claim 13, wherein the first end of the protrusion is at a bottom end of the protrusion, and wherein the U-shaped channel is below the first chamber and the second chamber.

15. The diagnostic cartridge of claim 14, wherein the bottom portion of the U-shaped channel is continuously concave and transitions from a first portion extending upward toward the first chamber to a second portion extending upward toward the second chamber without a horizontal segment therebetween.
